(19) United States
(12) Patent Application Publication
(10) Pub. No.: US 2024/0043369 A1
(43) Pub. Date: Feb. 8, 2024

(54) CROSSLINKING COMPOUNDS AND CROSSLINKED ACRYLIC POLYMERIC MATERIALS

(71) Applicants: Duke University, Durham, NC (US); Massachusetts Institute of Technology, Cambridge, MA (US)

(72) Inventors: Stephen L. Craig, Durham, NC (US); Jeremiah A. Johnson, Cambridge, MA (US); Shu Wang, Durham, NC (US); Michael Rubinstein, Durham, NC (US); Abraham Herzog-Arbeitman, Cambridge, MA (US)

(21) Appl. No.: 18/343,417

(22) Filed: Jun. 28, 2023

Related U.S. Application Data
(60) Provisional application No. 63/356,292, filed on Jun. 28, 2022.

(51) Int. Cl. C07C 69/757; C08F 36/20; C07C 69/753

(52) U.S. Cl. CPC C07C 69/757; C08F 36/20; C07C 69/753

(57) ABSTRACT

Disclosed herein are cyclobutane-based crosslinking compounds that, when incorporated into acrylate-based polymeric materials, can produce toughened acrylate polymer networks. Also disclosed herein are polymers comprising the crosslinkers, methods of preparing toughened polymer networks using the crosslinkers, and methods of using the polymer networks.

### CROSS-REFERENCE TO RELATED APPLICATIONS

This application claims the benefit of and priority to U.S. Provisional Patent Application No. 63/356,292, filed on Jun. 28, 2022, the contents of which are incorporated by reference herein in their entirety.

### STATEMENT REGARDING FEDERALLY SPONSORED RESEARCH

This invention was made with government support under CHE-2116298 awarded by the National Science Foundation. The government has certain rights in the invention.

### TECHNICAL FIELD

Disclosed herein are cyclobutane-based crosslinking compounds that, when incorporated into acrylate-based polymeric materials, can produce toughened acrylate polymer networks. Also disclosed herein are polymers comprising the crosslinkers, methods of preparing toughened polymer networks using the crosslinkers, and methods of using the polymer networks.

### BACKGROUND

The lifetime and utility of acrylate-based polymer networks are determined by their ability to stretch repeatedly without breaking. At sufficiently high strains, the network breaks by forming a crack that subsequently propagates through the material to the point of failure. Tearing at the macroscopic level is resisted at the molecular level by polymer chains within the network that need to break in order for the crack to propagate. The scission of covalent chains occurs through a chemical reaction—typically homolytic bond scission that is accelerated by the tension in overstretched chains at the propagating crack front.

### SUMMARY

It is possible to design and incorporate a small fraction of mechanically scissile functional groups (mechanophores) whose reactivity dominates polymer chain scission events. In such systems, the mechanical properties of the network might be expected to reflect the force-coupled reactivity of the mechanophores. When mechanophores are embedded into the middle of each elastically active network strand (FIG. 1A), the molecular and material properties are correlated in an intuitive manner: mechanochemical reactions that require less force to break on a given timescale result in weaker polymer networks (Wang et al. J. Am. Chem. Soc. 143, 3714-3718 (2021); Bowser et al. J. Am. Chem. Soc. 143, 5269-5276 (2021)). However, as demonstrated herein, when the mechanophore holds the network together through side-chain cross-linking in randomly cross-linked networks (FIG. 1B), the material properties depend on the force-coupled reactivity of the mechanophore.

Disclosed herein are cyclobutane-based mechanophore cross-linkers that break via force-triggered cycloreversion. When incorporated into acrylate-based polymers, these cross-linkers produce acrylate-based polymer networks that are up to 9 times tougher than conventional analogs. As demonstrated further in the Examples, the response is attributed to a combination of long, strong primary polymer strands and cross-linker scission forces that are ˜ 5-fold smaller than control cross-linkers at the same timescales. The enhanced toughness comes without the hysteresis associated with noncovalent cross-linking, and it is observed in multiple different acrylate elastomers, in fatigue as well as constant displacement rate tension, and in a gel as well as elastomers.

Accordingly, disclosed herein is an acrylate polymer comprising a moiety of formula (I) or a moiety of formula (II):

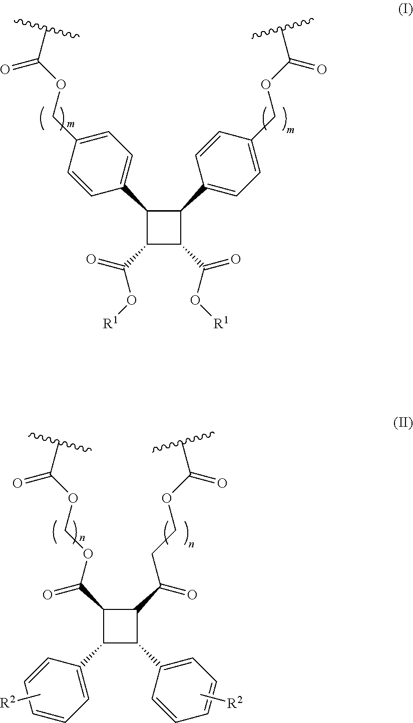

wherein:
R¹ is selected from C₁-C₁₂ alkyl and hydrogen;
R² is selected from hydrogen, halo, C₁-C₆ alkyl, C₁-C₆ haloalkyl, cyano, and nitro;
m is 1, 2, 3, 4, 5, or 6; and
n is 1, 2, 3, 4, 5, or 6.

In some embodiments, the acrylate polymer comprises a moiety of formula (I). In some embodiments, R¹ is methyl. In some embodiments, m is 4. In some embodiments, the moiety of formula (I) is:

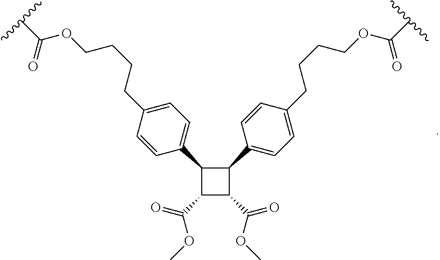 [SMILES: *C(=O)OCCCCc1ccc([C@@H]2[C@@H](C(=O)OC)[C@@H](C(=O)OC)[C@@H]2c2ccc(CCCCOC(*)=O)cc2)cc1]

In some embodiments, the acrylate polymer comprises a moiety of formula (II). In some embodiments, R² is bromo. In some embodiments, n is 4. In some embodiments, the moiety of formula (II) is:

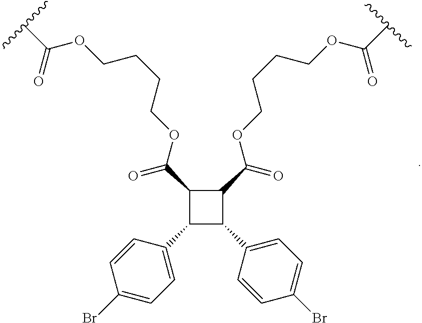

In some embodiments, the acrylate polymer comprises at least one (meth)acrylate monomer selected from an alkyl (meth)acrylate, a hydroxyalkyl (meth)acrylate, an alkoxyalkyl (meth)acrylate, a cycloalkyl (meth)acrylate, and an aromatic (meth)acrylate.

In some embodiments, the acrylate polymer comprises an alkyl (meth)acrylate monomer selected from methyl (meth)acrylate, ethyl (meth)acrylate, n-propyl (meth)acrylate, n-butyl (meth)acrylate, iso-butyl (meth)acrylate, tert-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, and stearyl (meth)acrylate.

In some embodiments, the acrylate polymer comprises a hydroxyalkyl (meth)acrylate monomer selected from hydroxymethyl (meth)acrylate, 2-hydroxyethyl (meth)acrylate, 3-hydroxypropyl (meth)acrylate, and 4-hydroxybutyl (meth)acrylate.

In some embodiments, the acrylate polymer comprises an alkoxyalkyl (meth)acrylate selected from 2-methoxyethyl (meth)acrylate, 2-ethoxyethyl (meth)acrylate, 1-methyl-2-methoxyethyl (meth)acrylate, ethylene glycol methyl ether (meth)acrylate, diethylene glycol methyl ether (meth)acrylate, and triethylene glycol methyl ether (meth)acrylate.

In some embodiments, the acrylate polymer comprises a cycloalkyl (meth)acrylate selected from cyclopentyl (meth)acrylate, cyclohexyl (meth)acrylate, 2-cyclohexylethyl (meth)acrylate, 3-cyclohexylpropyl (meth)acrylate, 2-norbornyl (meth)acrylate, and isobornyl (meth)acrylate.

In some embodiments, the acrylate polymer comprises at least two different (meth)acrylate monomers.

In another aspect, disclosed herein is a compound of formula (III) or formula (IV):

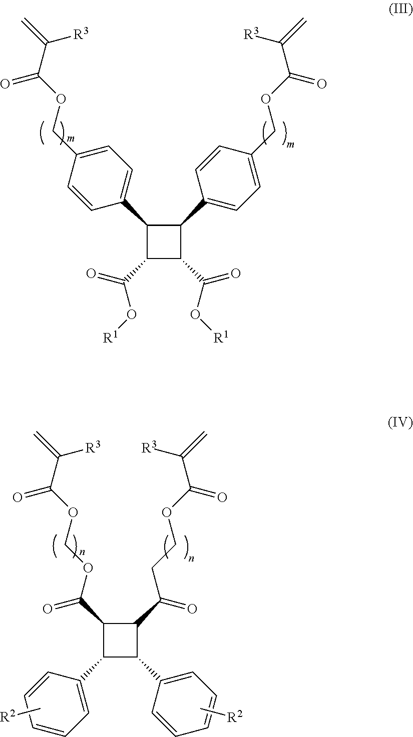

wherein:
R¹ is selected from C₁-C₁₂ alkyl and hydrogen;
R² is selected from hydrogen, halo, C₁-C₆ alkyl, C₁-C₆ haloalkyl, cyano, and nitro;
R³ is selected from hydrogen and methyl;
m is 1, 2, 3, 4, 5, or 6; and
n is 1, 2, 3, 4, 5, or 6.

In some embodiments, the compound is a compound of formula (III). In some embodiments, R¹ is methyl. In some embodiments, R³ is hydrogen. In some embodiments, m is 4. In some embodiments, the compound of formula (III) is:

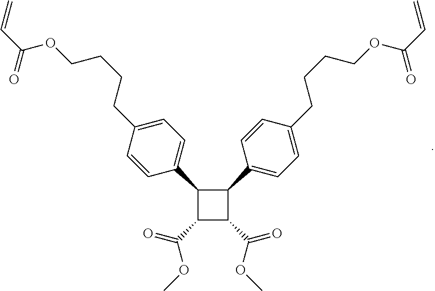 [SMILES: C=CC(=O)OCCCCc1ccc([C@@H]2[C@@H](C(=O)OC)[C@@H](C(=O)OC)[C@@H]2c2ccc(CCCCOC(=O)C=C)cc2)cc1]

In some embodiments, the compound is a compound of formula (IV). In some embodiments, R² is bromo. In some embodiments, R³ is hydrogen. In some embodiments, n is 4. In some embodiments, the compound of formula (IV) is:

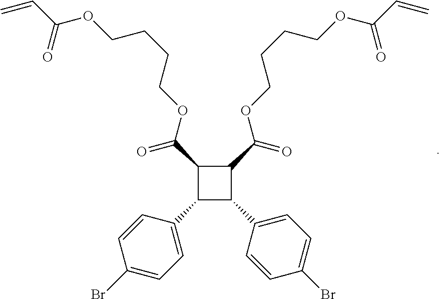

In another aspect, disclosed herein is an acrylate polymer prepared by:
(a) controlled radical polymerization of a first mixture comprising a (meth)acrylate monomer, to form a pre-gel mixture comprising acrylate polymer chains; and
(b) crosslinking the acrylate polymer chains in the pre-gel mixture with the compound of formula (III) or formula (IV).

In some embodiments, the controlled radical polymerization is reversible addition-fragmentation chain transfer (RAFT). In some embodiments, the first mixture further comprises a photoinitiator and a chain transfer agent. In some embodiments, the first mixture comprises at least two different (meth)acrylate monomers. In some embodiments, the compound of formula (III) or formula (IV) is:

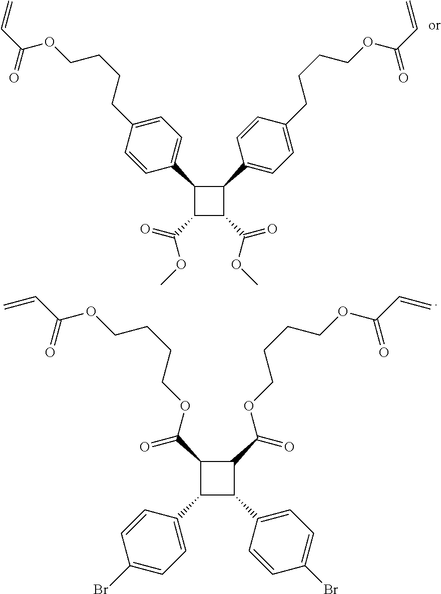

In another aspect, disclosed herein is a method of toughening an acrylate-based polymeric material, comprising:
incorporating a moiety of formula (I) or formula (II) into the acrylate-based polymeric material:

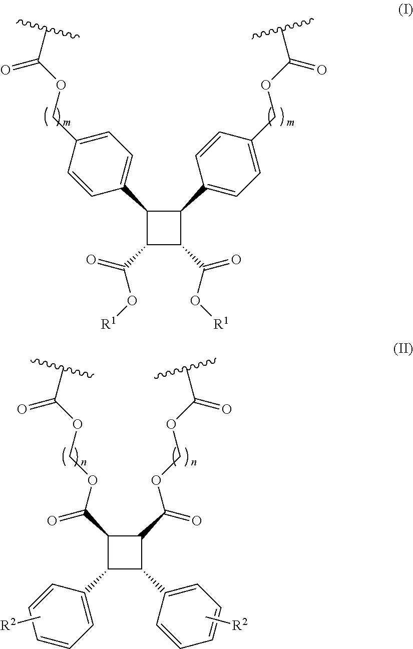

wherein:
R¹ is selected from C₁-C₁₂ alkyl and hydrogen;
R² is selected from hydrogen, halo, C₁-C₆ alkyl, C₁-C₆ haloalkyl, cyano, and nitro;
m is 1, 2, 3, 4, 5, or 6; and
n is 1, 2, 3, 4, 5, or 6.

In some embodiments, the incorporating step comprises:
(a) polymerizing a (meth)acrylate monomer to form a pre-gel mixture;
(b) adding a compound of formula (III) or formula (IV) to the pre-gel mixture to form a second mixture:

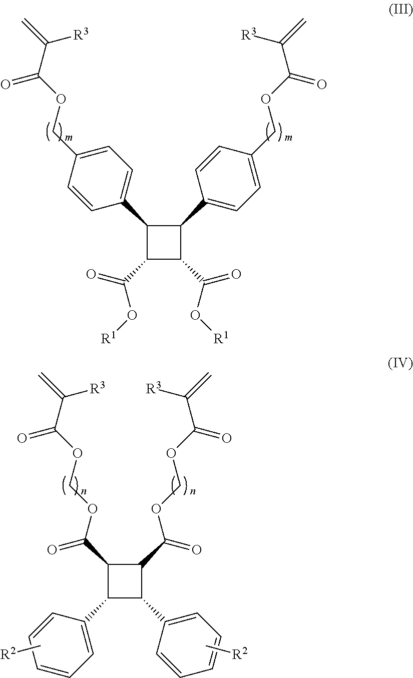

wherein:
   R¹ is selected from C₁-C₁₂ alkyl and hydrogen;
   R² is selected from hydrogen, halo, C₁-C₆ alkyl, C₁-C₆ haloalkyl, cyano, and nitro;
   R³ is selected from hydrogen and methyl;
   m is 1, 2, 3, 4, 5, or 6; and
   n is 1, 2, 3, 4, 5, or 6; and
(c) exposing the second mixture to light to incorporate the moiety of formula (I) or formula (II) into the acrylate-based polymeric material and form the toughened acrylate-based polymeric material.

In some embodiments, the polymerizing step comprises reversible addition-fragmentation chain transfer (RAFT) polymerization. In some embodiments, the polymerizing step comprises reacting the (meth)acrylate monomer with a photoinitiator and a chain transfer agent. In some embodiments, the polymerizing step comprises polymerizing a mixture of at least two different (meth)acrylate monomers.

Other aspects and embodiments of the disclosure will become apparent in light of the following description and the drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show network designs and the mechanochemical reactivities of cross-linkers. Mechanophores embedded (A) into the middle of chains, and (B) as the side-chain cross-linkers of primary chains. (C) General procedure for RAFT network preparation. (D) Force-triggered cycloreversion of C1. (E) Lifetime as a function of breaking force for C1 and for a common C—C bond. Dashed lines are shown as guides

FIG. 2 shows a mold setup. A “sandwich” mold was made by binding two glass plates and a Buna-N rubber (˜0.8 mm thick) spacer together. Inner dimension of the spacer is around 60×120 mm (120 mm×120 mm for the samples for fatigue tests). Low friction transparent FEP tape (˜0.0035″ thick) and PTFE tape (˜0.012″ thick) were applied to the glass plates to help with demolding.

FIGS. 3A-3F show results of frequency sweeps. Elastomers with stoichiometry of (A) [M]: [C]: [CTA]: [PI]=1:1/50:1/300:1/1500, (B) [M]: [C]: [CTA]: [PI]=1:1/50:1/3600:1/6000, (C) [M]: [C]: [CTA]: [PI]=1:1/100:1/1200:1/2000, (D) [M]: [C]: [CTA]: [PI]=1:1/200:1/1200:1/2000. (E) Gels (50% volume fraction) with a stoichiometry of [M]: [C]: [PI]=1:1/200:1/4500. (F) Highly entangled polymer with a stoichiometry of [M]: [PI]=1:10⁻⁶. No additional crosslinker or solvent was used. Entanglement modulus was taken from the G′ at 0.1 Hz in fig. F. Error bars are standard deviation.

FIGS. 4A-4D show sol fractions and equilibrium swelling ratios. (A) Sol fractions and (B) equilibrium swelling ratios Q of elastomers with [C]: [M]=1:50, and Nₚ≈350, 1400, 2000, respectively. (C) Sol fractions and (D) equilibrium swelling ratios Q of elastomers with Nₚ≈1400, [C]: [M]=1:200, 1:100, 1:50. Error bars are standard deviation.

FIG. 5 shows DSC measurements of E1 and E2 (Exo up). The glass transition temperature of E1 is about 4 degrees Celsius higher than that of E2, but both are well below room temperature, at which mechanical tests were performed.

FIG. 6 shows stress-strain curves for different geometries. Columns are different sample geometries: high rectangle (tensile test), wide rectangle (pure shear), and notched wide rectangle. Rows are the same samples tested under these geometries. The information of tested samples is shown at the beginning of each row. For each plot, the y-axis is the stress in kPa, the x-axis is the strain in percent.

FIG. 7 shows a sample setup for fatigue tests. Two acrylic sheets on each side were used to achieve a wide and firm grip of the elastomer film. Buna-N rubber was used between two acrylic sheets as a spacer.

FIGS. 8A-8H show mechanical characterizations for elastomers. (A) Frequency sweeps (×3 samples each) (strain=0.5%), uniaxial extension stress-strain curves of (B) unnotched (×3 samples each) and (C) notched (×5 samples each) samples in pure shear geometry, and (D) tearing energies for E1 and E2; p=6.5×10⁻⁶, T-test assuming equal variance. (E) Uniaxial tensile test of notched samples in pure shear geometry and (F) tearing energies for NW-1 and NW-2 adapted from previous work. Crack growth per cycle as a function of energy release rate (×3 samples each) for (G) E1 and (H) E2. Error bars are standard deviations.

FIGS. 9A-9E show thermal stability of E1 and its cyclobutane crosslinks. (A) E1 was submerged in DMF and reached equilibrium swelling. (B) E1 was heated to 100° C. in DMF solvent for 96 hours and cooled down to room temperature. No further swelling was observed, indicating cyclobutane crosslinks remain stable under this condition. (C) TGA of compound 1k. The temperature was ramped from 25° C. to 800° C. with a rate of 20° C./min. The onset of significant weight loss is around 260° C. (D) DSC of compound 1k (Exo up). The temperature was ramped from 20° C. to 300° C. with a rate of 10° C./min for both heating cycles. (E) NMR spectra of compound 1k in DMSO-d₆ (black), and after heating at 150° C. for 24 hours (red).

FIGS. 10A-10B show SMFS characterization of P1. (A) A representative force-displacement curve for P1. (B) Reaction rate-force dependence of P1 extracted from constant velocity experiment. The dashed line is the linear fitting of the logarithm of the reaction rate as a function of force.

FIGS. 11A-11C show stress-strain curves of un-notched and notched gels. Gels were prepared at 50% volume fraction (propylene carbonate as solvent) with a stoichiometry of [M]: [C]: [PI]=1:1/200:1/4500 without CTA. (A) Tensile test of pure shear specimens. (B) Tearing test of pure shear specimens. (C) Tearing energies comparison.

FIGS. 12A-12C show stress-strain curves of un-notched and notched ethyl acrylate elastomer. Elastomers were prepared with a stoichiometry of [M]: [C]: [CTA]: [PI]=1:1/200:1/1200:1/2000. Ethyl acrylate was used as the monomer instead of 2-methoxyethyl acrylate. (A) Tensile test of pure shear specimens. (B) Tearing test of pure shear specimens. (C) Tearing energies comparison.

FIGS. 13A-13F show a toughening mechanism of mechanochemically weak cross-linkers. (A) Mechanochemically weak cross-linkers break preferentially before mechanically strong primary chains break in E1, which increases the tortuosity of the crack path. (B) Cross-linkers that are similarly strong as the primary chains in E2 do not break preferentially. Cartoons are schematic only. (C) Storage moduli (×3 samples each) and (D) tearing energies (×5 samples each) of E1 and E2 with different Nₚ. (E) Storage moduli of E1 and E2 with different crosslinking densities (F) Log-log plot of I′ vs. G′ of E1 and E2 with different crosslinking densities. Dashed lines are power-law fittings. Error bars are standard deviations.

FIG. 14 shows the number of bonds broken in the network. Red curves represent the strong crosslinker network and blue curves represent the weak crosslinker network. Solid lines—broken primary chain bonds and dashed lines—broken crosslinkers. The blue solid line coincides with horizontal axis as no primary bonds are broken in the weak crosslinker network, while in the strong crosslinker network most broken bonds belong to primary chains.

FIG. 15 shows the increase of average neighboring strand length per scission event at strain of 6. Red column represents the strong crosslinker network and blue column represents the weak crosslinker network.

FIGS. 16A-16E show cyclic loading of E1. (A) Cyclic stress-strain curves for E1 at maximum strains of 0.7 (8 cycles), 0.73 (8 cycles), 0.76 (6 cycles), and 0.79 (2 cycles). The curves are intentionally offset for visual clarity. Strain rate was set at 2.5% s⁻¹. No significant hysteresis was found for these maximum strains. Young's modulus versus cycle number at different maximum strains: (B) 0.7, (C) 0.73, (D) 0.76, and (E) 0.79 (sample broke during the loading of 3^{rd} cycle). The cyclic loading test was performed on the same sample until it breaks abruptly. No decrease in modulus was found after several loadings. As the applied strain approaches the strain at break (˜80%), the number of cycles the network can sustain reduces.

FIG. 17 shows synthesis of PA1, E4 and PA2. Elastomers with C4 were prepared with four stoichiometries, [M]: [CTA]=400:1, 600:1, 1200:1, and 3600:1. Four corresponding PA1 were prepared as well. [M]: [C] was fixed at 50:1.

FIGS. 18A-18C show elastomer E3 made from intermediate crosslinker C3. Elastomers were prepared with a stoichiometry of [M]: [C]: [CTA]: [PI]=1:1/50:1/1200:1/2000. (A) Force-trigger cycloreversion of “intermediate” crosslinker C3. (B) Frequency sweep of E1-3. (C) Tearing energies of E1-3. The T-test between E2 and E3 shows a p-value of p=0.089>0.05 (T-test assuming equal variance), indicating the tearing energy of E3 is not significantly larger than E2.

FIGS. 19A-19D show elastomer E5 made from crosslinker C5. Elastomer E5 were prepared with a stoichiometry of [M]: [C]: [CTA]: [PI]=1:1/50:1/1200:1/2000. (A) Frequency sweep comparison for E1, E2, and E5 (×3 samples). (B) Uniaxial tensile stress-strain curves comparison for E1, E2, and E5 (×3 samples). (C) Notched stress-strain curves comparison for E1, E2, and E5 (×5 samples). (D) Tearing energies of E1, E2, and E5 (16.8±7.2 J·m⁻²). The T-test between E2 and E5 shows a p-value ofp=0.22>0.05 (T-test assuming equal variance), indicating the tearing energy of E5 is not significantly different from that of E2.

FIG. 20 shows GPC traces for PA1 and PA2. PA1 are shown in solid lines and PA2 are shown in dashed lines. Numbers and colors indicate the different ratios [M]/[CTA] used during preparation.

FIG. 21 shows GPC traces for Nₚ estimation. For [M]:[CTA]=300 and 1200 (blue and red), the corresponding PA1 (solid lines) were used to estimate Nₚ. For [M]:[CTA]=3600 (green), the corresponding PA2 (dashed line) was used to estimate Nₚ.

FIG. 22 shows bond potentials used in simulations. The FENE bond potential (black curve) is used for equilibrating melts and networks. The Morse potential (red for strong bonds and blue for weak bonds) is used for pulling simulations where bond rupture is desired.

FIGS. 23A-23C show schematic illustrations for different scenarios when breaking a crosslinker. Strands colored in red are the neighboring strands associated with breaking a crosslinker. Strands in blue color represent elastically inactive strands. These structures are local structures only. Different strands could belong to the same primary chain.

FIGS. 24A-24C show schematic illustrations for different scenarios when breaking a primary chain bond. Strands colored in red are the neighboring strands associated with breaking a crosslinker. Strands in blue color represent elastically inactive strands. These structures are local structures only. Different strands could belong to the same primary chain.

FIG. 25 shows the number of elastically active and elastically inactive strands as the network is uniaxially stretched. The red curves represent the data for the strong crosslinker network (E2), and the blue curves represent the data for the weak crosslinker network (E1).

FIGS. 26A-26B show a comparison between intramolecular and intermolecular crosslinks. Number (A) and the fraction (B) of broken intramolecular and intermolecular crosslinks as a function of strain. Red curves are networks made from strong crosslinkers, while blue curves are networks made from weak crosslinkers.

### DETAILED DESCRIPTION

Disclosed herein are cyclobutane-based crosslinking compounds that, when incorporated into acrylate-based polymeric materials, can produce toughened acrylate polymer networks. Also disclosed herein are polymers comprising the crosslinkers, methods of preparing toughened polymer networks using the crosslinkers, and methods of using the crosslinked polymers.

### A. Definitions

Unless otherwise defined, all technical terms used herein have the same meaning as commonly understood by one of ordinary skill in the art to which this disclosure belongs.

Definitions of specific terms, including certain functional groups and chemical terms, are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkoxy” refers to an alkyl group, as defined herein, appended to the parent molecular moiety through an oxygen atom. Representative examples of alkoxy include, but are not limited to, methoxy, ethoxy, n-propoxy, isopropoxy, n-butoxy, and tert-butoxy.

As used herein, the term “alkoxyalkyl” refers to an alkyl group, as defined herein, in which at least one hydrogen atom (e.g., one hydrogen atom) is replaced with an alkoxy group, as defined herein. Representative examples of alkoxyalkyl include, but are not limited to, methoxymethyl, 2-methoxyethyl, and 2-ethoxyethyl.

As used herein, the term “aryl” refers to a radical of a monocyclic, bicyclic, or tricyclic 4n+2 aromatic ring system (e.g., having 6, 10, or 14π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl). In some embodiments, an aryl group has fourteen ring carbon atoms (“C₁₄ aryl,” e.g., anthracenyl and phenanthrenyl).

As used herein, the term “cyano” refers to a group —CN.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halo” or “halogen” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “hydroxy” refers to an —OH group.

As used herein, the term “hydroxyalkyl” refers to an alkyl group, as defined herein, in which at least one hydrogen atom (e.g., one hydrogen atom) is replaced with a hydroxy group. Representative examples of hydroxyalkyl include, but are not limited to, hydroxymethyl, 2-hydroxyethyl, 3-hydroxypropyl, and 4-hydroxybutyl.

As used herein, the term “(meth)” designates optional methyl substitution. Thus, a term such as “(meth)acrylates” denotes both methacrylates and acrylates. For example, the term “methyl (meth)acrylate” refers to methyl acrylate and methyl methacrylate.

As used herein, the term “nitro” refers to a group —NO₂.

### B. Crosslinkers and Polymers

Disclosed herein are crosslinking compounds, including compounds of formula (III) and compounds of formula (IV):

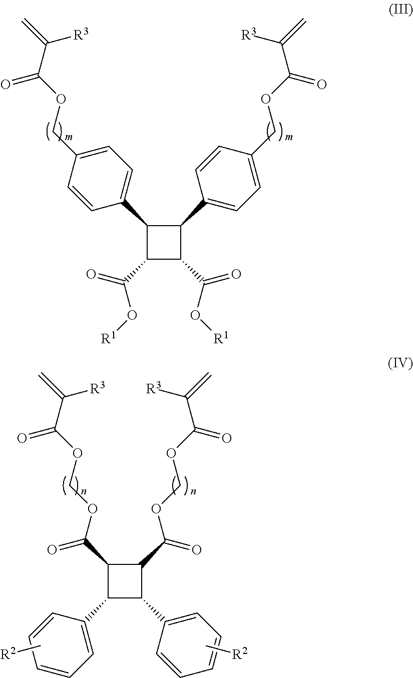

wherein:
R¹ is selected from C₁-C₁₂ alkyl and hydrogen;
R² is selected from hydrogen, halo, C₁-C₆ alkyl, C₁-C₆ haloalkyl, cyano, and nitro;
R³ is selected from hydrogen and methyl;
m is 1, 2, 3, 4, 5, or 6; and
n is 1, 2, 3, 4, 5, or 6.

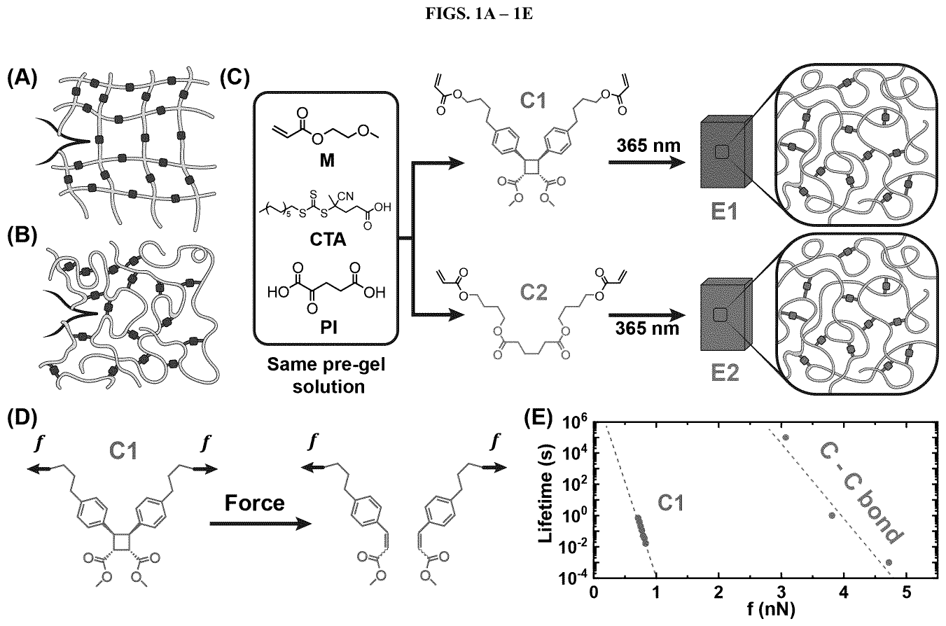

The compounds of formula (III) and compounds of formula (IV) include a cyclobutane moiety. For example, these compounds include a cis-diaryl substituted cyclobutane-based mechanophore. When incorporated into polymeric materials, these mechanophores react under tension by means of a force-coupled [2+2] cycloreversion to form two cinnamates (see, e.g., FIG. 1D).

In some embodiments, the compound is a compound of formula (III). In some embodiments, R¹ is C₁-C₁₂ alkyl. In some embodiments, R¹ is methyl. In some embodiments, R³ is hydrogen. In some embodiments, R³ is methyl. In some embodiments, m is 1. In some embodiments, m is 2. In some embodiments, m is 3. In some embodiments, m is 4. In some embodiments, m is 5. In some embodiments, m is 6. In some embodiments, the compound of formula (III) is:

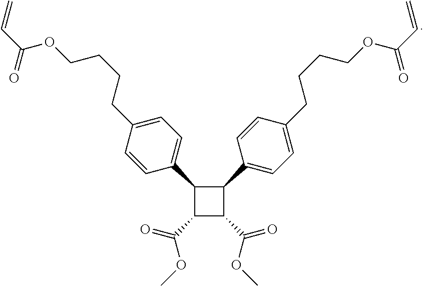 [SMILES: C=CC(=O)OCCCCc1ccc([C@@H]2[C@@H](C(=O)OC)[C@@H](C(=O)OC)[C@@H]2c2ccc(CCCCOC(=O)C=C)cc2)cc1]

In some embodiments, the compound is a compound of formula (IV). In some embodiments, R² is halo. In some embodiments, R² is bromo. In some embodiments, R¹ is methyl. In some embodiments, R³ is hydrogen. In some embodiments, R³ is methyl. In some embodiments, n is 1. In some embodiments, n is 2. In some embodiments, n is 3. In some embodiments, n is 4. In some embodiments, n is 5. In some embodiments, n is 6. In some embodiments, the compound of formula (III) is:

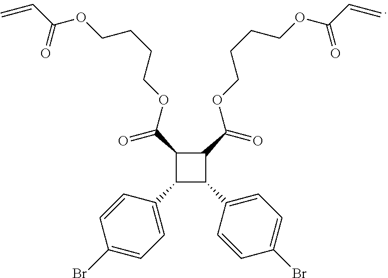

Crosslinking compounds of formula (III) and/or formula (IV) can be incorporated into acrylate-based polymers to crosslink such polymers. Once incorporated into an acrylate-based polymer, the polymer (i.e., a crosslinked acrylate polymer) will include a moiety of formula (I) or a moiety of formula (II):

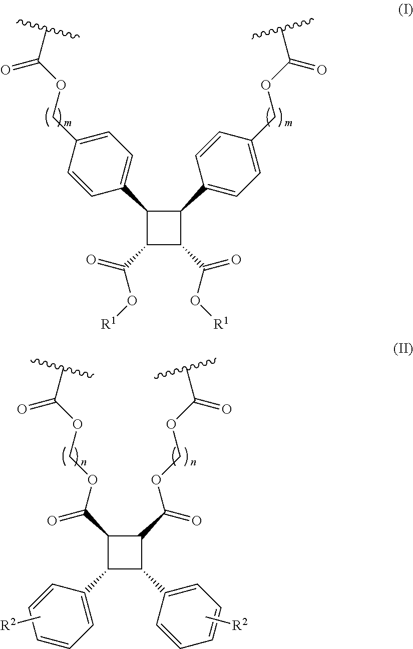

wherein:
R¹ is selected from C₁-C₁₂ alkyl and hydrogen;
R² is selected from hydrogen, halo, C₁-C₆ alkyl, C₁-C₆ haloalkyl, cyano, and nitro;
m is 1, 2, 3, 4, 5, or 6; and
n is 1, 2, 3, 4, 5, or 6.

In some embodiments, the when the compound of formula (III) is used as the crosslinker, the polymer will comprise a moiety of formula (I). In some embodiments, R¹ is C₁-C₁₂ alkyl. In some embodiments, R¹ is methyl. In some embodiments, R³ is hydrogen. In some embodiments, R³ is methyl. In some embodiments, m is 1. In some embodiments, m is 2. In some embodiments, m is 3. In some embodiments, m is 4. In some embodiments, m is 5. In some embodiments, m is 6. In some embodiments, the moiety of formula (I) is:

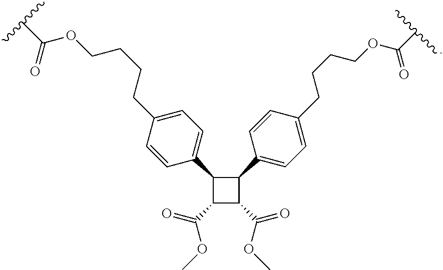 [SMILES: *C(=O)OCCCCc1ccc([C@@H]2[C@@H](C(=O)OC)[C@@H](C(=O)OC)[C@@H]2c2ccc(CCCCOC(*)=O)cc2)cc1]

In some embodiments, when the compound of formula (IV) is used as the crosslinker, the polymer will comprise a moiety of formula (II). In some embodiments, R² is halo. In some embodiments, R² is bromo. In some embodiments, R¹ is methyl. In some embodiments, R³ is hydrogen. In some embodiments, R³ is methyl. In some embodiments, n is 1. In some embodiments, n is 2. In some embodiments, n is 3. In some embodiments, n is 4. In some embodiments, n is 5. In some embodiments, n is 6. In some embodiments, the moiety of formula (II) is:

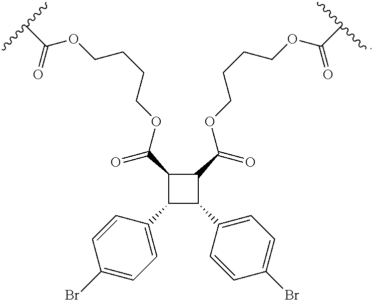

The acrylate polymers described herein can include one or more (meth)acrylate monomers. Any (meth)acrylate monomers can be used. In some embodiments, the polymers include at least one (meth)acrylate monomer selected from an alkyl (meth)acrylate, a hydroxyalkyl (meth)acrylate, an alkoxyalkyl (meth)acrylate, a cycloalkyl (meth)acrylate, and an aromatic (meth)acrylate.

For example, in some embodiments, the polymer comprises an alkyl (meth)acrylate monomer. Exemplary alkyl (meth)acrylates include, but are not limited to, methyl (meth)acrylate, ethyl (meth)acrylate, n-propyl (meth)acrylate, n-butyl (meth)acrylate, iso-butyl (meth)acrylate, tert-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, and stearyl (meth)acrylate.

In some embodiments, the polymer comprises a hydroxyalkyl (meth)acrylate monomer. Exemplary hydroxyalkyl (meth)acrylate monomers include, but are not limited to, hydroxymethyl (meth)acrylate, 2-hydroxyethyl (meth)acrylate, 3-hydroxypropyl (meth)acrylate, and 4-hydroxybutyl (meth)acrylate.

In some embodiments, the polymer comprises an alkoxyalkyl (meth)acrylate monomer. Exemplary alkoxyalkyl (meth)acrylate monomers include, but are not limited to, 2-methoxyethyl (meth)acrylate, 2-ethoxyethyl (meth)acrylate, 1-methyl-2-methoxyethyl (meth)acrylate, ethylene glycol methyl ether (meth)acrylate, diethylene glycol methyl ether (meth)acrylate, and triethylene glycol methyl ether (meth)acrylate.

In some embodiments, the polymer comprises a cycloalkyl (meth)acrylate monomer. Exemplary cycloalkyl (meth)acrylate monomers include, but are not limited to, cyclopentyl (meth)acrylate, cyclohexyl (meth)acrylate, 2-cyclohexylethyl (meth)acrylate, 3-cyclohexylpropyl (meth)acrylate, 2-norbornyl (meth)acrylate, and isobornyl (meth)acrylate.

The polymers can include at least two different (meth)acrylate monomers, such as any of the monomers described herein. For polymer can be a block copolymer (e.g., a diblock copolymer or a triblock copolymer), a random copolymer, a graft copolymer, or the like.

The crosslinked acrylate polymers described herein can be prepared by a number of methods, including controlled radical polymerization methods, such as reversible addition fragmentation chain transfer polymerization (RAFT), atom transfer radical polymerization (ATRP), stable free radical polymerization (SFRP), nitroxide-mediated polymerization (NMP), and the like. In some embodiments, the acrylate polymers are prepared by reversible addition-fragmentation chain transfer (RAFT).

For example, in some embodiments, the acrylate polymer can be prepared by:
(a) controlled radical polymerization (e.g., RAFT) of a (meth)acrylate monomer, to form a pre-gel mixture comprising acrylate polymer chains; and
(b) crosslinking the acrylate polymer chains in the pre-gel mixture with the compound formula (III) or a compound of formula (IV).

The controlled radical polymerization (e.g., RAFT) reaction is conducted using an initiator, which is an agent capable of producing a free radical. In some embodiments, the controlled radical polymerization (e.g., RAFT) is conducted using a photoinitiator. An initiator is a compound that decomposes into radicals which subsequently react with a monomer to initiate a free-radical polymerization reaction. Photoinitiators decompose by photochemical processes. Typical examples of photoinitiators include, but are not limited to: benzil, benzoin, acetophenone, benzophenone, camphorquinone, or derivatives thereof, monoacyl and bisacyl phosphine oxides; and α-ketoesters including α-ketoglutaric acid, ethyl pyruvate, and the like. Combinations of photoinitiators can also be used. In some embodiments, the photoinitiator is an α-ketoester. In some embodiments, the photoinitiator is α-ketoglutaric acid.

In some embodiments, the controlled radical polymerization (e.g., RAFT) is conducted using a chain transfer agent. Representative chain transfer agents include, but are not limited to: trithiocarbonates such as 3,5-bis(2-dodecylthiocarbonothioylthio-1-oxopropoxy) benzic, 3-butenyl 2-(dodecylthiocarbonothioylthio)-2-methyl propionate, 2-(2-carboxyethylsulfanylthiocarbonylsulfanyl)-proprionic acid, 4-((((2-carboxyethyl)thio) carbonothioyl)thio)-4-cyanopentanoic acid, 2-cyanobutan-2-yl 4-chloro-3,5-dimethyl-1H-pyrazole-1-carbodithioate, 2-cyanobutanyl-2-yl 3,5-di methyl-1H-pyrazole-1-carbodithioate, 4-cyano-4-[(dodecylsulfanylthiocarbonyl)sulfanyl]pentanoic acid, 2-(butylthiocarbonothioylthio)propanoic acid, 4-cyano-4-(ethylcarbonothioylthio) pentanoic acid, 4-cyano-4-[(dodecylsulfanylthiocarbonyl)sulfanyl]pentanol, cyanomethyl (3,5-Dimethyl-1H-pyrazole)-carbodithioate, cyanomethyl dodecyl trithiocarbonate, cyanomethyl [3-(trimethoxysilyl)propyl] trithiocarbonate, 2-cyano-2-propyl dodecyl trithiocarbonate, S,S-di benzyl trithiocarbonate, 2-(dodecyl-thiocarbonothioylthio)-2-methylpropionic acid, 2-(dodecyl-thiocarbonothioyl-thio)-2-methylpropionic acid, 3-azido-1-propanol ester, 2-(dodecylthiocarbonothioylthio)-2-methylpropionic acid, N-hydroxysuccinimide ester of 4-cyano-4-[(dodecylsulfanylthiocarbonyl)sulfanyl]pentanoic acid, pentafluorophenyl ester of 2-(dodecylthiocarbonothioylthio)-2-methylpropionic acid, 2-(dodecylthiocarbonothioylthio)propionic acid, methyl 2-(dodecyl)-2-methylpropionate, pentaerythritol tetrakis[2-(dodecylthiocarbonothioylthio)-2-methylpropionate], phthalimidomethyl butyl trithiocarbonate, poly(acrylic acid) having a 2-(dodecylthiocarbonothioylthio)-2-methylpropionic acid end, poly(ethylene glycol)bis[2-(dodecylthiocarbonothioylthio)-2-methylpropionate], poly(ethylene glycol) methyl ether 4-cyano-4-[(dodecylsulfanylthiocarbonyl)sulfanyl]pentanoate, poly(ethylene glycol) methyl ether (4-cyano-4-pentanoate dodecyl trithiocarbonate), poly(ethylene glycol)methyl ether (4-cyano-4-pentanoate dodecyl trithiocarbonate), poly (ethylene glycol)methyl ether (4-cyano-4-pentanoate dodecyl trithiocarbonate), poly(ethylene glycol) methyl ether 2-(dodecylthiocarbonothioylthio)-2-methylpropionate, poly(ethylene glycol)methyl ether 2-(dodecylthiocarbonothioylthio)-2-methylpropionate, poly(ethylene glycol)methyl ether (2-methyl-2-propionic acid dodecyl trithiocarbonate)L-lactide) 4-cyano-4-[(dodecylsulfanyl-thiocarbonyl)sulfanyl] pentonate, poly(L-lactide) 4-cyano-4-[(dodecylsulfanyl-thiocarbonyl)sulfanyl] pentonate, poly(D,L-lactide), 4-cyano-4-[(dodecylsulfanyl-thiocarbonyl)sulfanyl] pentonate, polystyrene with an end of 2-(dodecylthiocarbonothioylthio)-2-methylpropionic acid. or 1,1,1-tris[(dodecylthiocarbonothioylthio)-2-methylpropionate] ethane; dithiocarbamates such as benzyl 1H-pyrrole-1-carbodithioate, cyanomethyl diphenylcarbamodithioate, cyanomethyl methyl(phenyl)carbamodithioate, cyanomethyl methyl(4-pyridyl)carbamodithioate, 2-cyanopropan-2-yl N-methyl-(pyridin-4-yl)carbamodithioate, methyl 2-[methyl(4-pyridinyl)carbamothioylthio] propionate, 1-succinimidyl-4-cyano-4-[N-methyl-N-(4-pyridyl)carbamothioylthio] pentanoate; dithioabenzoates such as benzyl benzodithioate, cyanomethyl benzodithioate, 4-cyano-4-(phenylcarbonothioylthio)pentanoic acid, N-succinimidyl ester of 4-cyano-4-(phenylcarbonothioylthio)pentanoic acid, 2-cyano-2-propyl benzodithioate, 2-cyano-2-propyl4-cyanobenzodithioate, ethyl 2-(4-methoxyphenylcarbonothioylthio)acetate, ethyl 2-methyl-2-(phenylthiocarbonylthio)propionate, ethyl 2-(phenylcarbonothioylthio)-2-phenylacetate, ethyl 2-(phenylcarbonothioylthio) propionate, 1-(methoxycarbonyl)ethyl benzodithioate, 2-(4-methoxyphenylcarbonothioylthio) ethanoic acid, 2-nitro-5-(2-propynyloxy)benzyl 4-cyano-4-(phenylcarbonothioylthio)pentanoate, 2-(phenylcarbonothioylthio) propanoic acid, or 2-phenyl-2-propyl benzodithioate; and switchable RAFT agents such as cyanomethyl methyl(4-pyridyl)carbamodithioate, 2-cyanopropan-2-yl N-methyl-N-(pyridin-4-yl)carbamodithioate, methyl 2-[methyl(4-pyridinyl)carbamothioylthio]propionate, or 1-succinimidyl-4-cyano-4-[N-methyl-N-(4-pyridyl) carbamothioylthio] pentanoate. In some embodiments, the chain transfer agent is 4-cyano-4-[(dodecylsulfanylthiocarbonyl)sulfanyl]pentanoic acid.

### C. Methods of Use

The crosslinking compounds disclosed herein are mechanically weak, yet produce acrylate polymers that have improved strength and tear resistance. Accordingly, disclosed herein is a method of toughening an acrylate-based polymeric material, comprising incorporating a moiety of formula (I) or formula (II) into the acrylate-based polymeric material. The moiety of formula (I) or formula (II) can be incorporated, for example, by crosslinking an acrylate polymer with a compound of formula (III) or a compound of formula (IV).

The toughened acrylate polymer networks can be used in any application in which it would be useful or desirable to use a mechanically strong polymeric material. For example, acrylate-based polymeric materials are often used in additive manufacturing processes (i.e., 3D printing), in materials used in artificial joints, and in cosmetic applications such as nail polish.

### EXAMPLES

Materials and Methods

Materials. Lab general solvents (dichloromethane, acetonitrile, hexane, ethyl acetate, acetone, tetrahydrofuran, dioxane, methanol, dimethyl formaldehyde) were purchased from VWR or Sigma Aldrich. Hydrogen was purchased from Airgas. 4-bromo-cinnamic acid, 4-(dimethylamino)pyridine (DMAP), 3-(3-dimethylaminopropyl)-1-ethyl-carbodiimide hydrochloride (EDC·HCl), copper (I) iodide (CuI), Sodium iodide (NaI), trans-N,N-dimethylcyclohexane-1,2-diamine, 3-Butyn-1-ol, bis(triphenylphosphine)palladium(II) dichloride, diisopropylamine, palladium on carbon (10 wt %), acrylic acid, 4-hydroxybutyl acrylate, adipic acid, 2-hydroxyethyl acrylate, pyridinium p-toluenesulfonate (PPTS), 2-methoxypropene, triethylamine (TEA), Grubbs II catalyst, 4-pentenoic anhydride, 9-oxabicyclo[6.1.0]non-4-ene, ethyl acrylate, 2-methoxylethyl acrylate, 4-Cyano-4-[(dodecylsulfanylthiocarbonyl)sulfanyl]pentanoic acid, α-ketoglutaric acid, and propylene carbonate were purchased from Sigma Aldrich, or Alfa Aesar, or ChemImpex, or Ambeed Inc., and used without further purification. Borosilicate glass plates, low friction transparent FEP tape (0.0035″ thick), low friction Polytetrafluoroethylene (PTFE) tape (0.012″ thick) was purchased from McMASTER-CARR.

General Methods. ¹H NMR and ¹³C NMR spectra were collected on a 500 MHz Bruker spectrometer. ¹H shifts are reported as chemical shift, multiplicity, coupling constant if applicable, and relative integral. Multiplicities are reported as: singlet (s), doublet (d), doublet of doublets (dd), doublet of triplets (dt), doublet of doublet of doublets (ddd), doublet of doublet of triplets (ddt), triplet (t), triplet of doublets (td), quartet (q), multiplet (m), or broad (br). High-resolution mass spectra were collected on an Agilent LCMS-TOF-DART at Duke University's Mass Spectrometry Facility.

Flash chromatography was performed using Silicycle SiliaFlash@ F60 gel (40-63 μm particle size, 230-400 mesh) and medium pressure liquid chromatography (MPLC) was performed on a Teledyne ISCO CombiFlash Rf 200.

Thermogravimetric analysis (TGA) was performed on a TA instrument (TGA 550) using 100 μL open platinum pans under nitrogen flow. A heating rate of 20° C./min. from 25° C. to 800° C. was utilized to obtain volatility and decomposition temperatures. Differential scanning calorimetry (DSC) was performed on a TA instrument (DSC2500). The sample of 4˜8 mg was placed in a non-hermetic Tzero aluminum pan and an empty pan was used as a reference pan. The DSC experiment was performed in a heat-cool cycle. Detailed parameters used for different experiments are mentioned herein.

Gel permeation chromatography (GPC) was performed on in-line two columns (Agilent PLgel 105 Å, 7.5×300 mm, 5 μm, part number PL1110-6550) at room temperature using inhibitor free THE at a flow rate of 1.0 mL/min. The flow rate was set using an Agilent 1260 Infinity Isocratic pump, molecular weights were calculated using in line Wyatt Optilab T-rEX refractive index detector and Wyatt miniDAWN TREOS multiangle light scattering detector, and UV absorbance was measured with an in-line Agilent 1260 Infinity UV detector. The UV detector monitored 190 to 800 nm with step of 2.0 nm and slit width of 4.0 nm. The refractive index increment (dn/dc) values were determined by using on-line 100% mass recovery assumption calculations built into Wyatt Astra software using injections of known concentration and mass. Before GPC analysis, 1-2 mg/mL in THE solutions were filtered through a 0.2 μm pore size PTFE syringe filters.

The AFM pulling experiments were conducted in toluene at an ambient temperature (˜23° C.) in the same manner as described previously (Wu et al. J. Am. Chem. Soc. 132, 15936-15938 (2010); Klukovich et al. Nat. Chem. 5, 110-114 (2013); Wang et al. Nat. Chem. 7, 323-327 (2015); Wang et al. J. Org. Chem. 80, 11895-11898 (2015); Kouznetsova et al. ChemPhysChem 18, 1486-1489 (2017)) using a homemade AFM, which was constructed using a Bruker (previously Digital Instruments) Multimode AFM head mounted on top of a piezoelectric positioner (Physik Instrumente, GmbH), similar to the one described in detail previously (Kouznetsova 2017). Sharp Microlever silicon probes (MSNL) were purchased from Bruker (Camarillo, CA) and the force curves used for analysis were obtained with rectangular-shaped cantilevers (205 μm×15 μm, nominal tip radius˜2 nm, nominal spring constant k˜0.02 N/m, frequency ˜15 kHz). Multiple probes of the same type were used throughout the course of the experiments. The spring constant of each cantilever was calibrated in air, using the thermal noise method, based on the energy equipartition theorem as described previously (Oberhauser et al. Nature 393, 181-185 (1998)). Cantilever tips were prepared by soaking in piranha solution for ˜15 min at room temperature. Silicon surfaces were prepared by soaking ˜30 min in hot piranha solution, followed by washing with DI-water and drying under a stream of nitrogen. The surface and cantilever were then placed in a UVO cleaner (ozone produced through UV light) for 15 min. After ozonolysis, the cantilever was mounted and ˜20 μL of a ˜0.1-0.05 mg mL⁻¹ polymer solution was added to the silicon surface and allowed to dry. Measurements were carried out in a fluid cell with scanning set for a series of constant velocity approaching/retracting cycles.

Rheological measurements were conducted on an Anton Paar MCR 302 rheometer with a 8 mm parallel plate geometry. Uniaxial tensile tests and tearing energy measurements were performed on a TA Instruments RSA III Dynamic Mechanical Analyzer (force resolution: 0.0001 N, displacement resolution: 1 m) at Duke University's Shared Material Instrument Facility (SMIF). Fatigue tests were performed on a Test Resources 910LX25 dynamic & fatigue test machine (force resolution: 0.0001 N, displacement resolution: 1 m).

### Example 1

### Compound Syntheses

### Synthesis of C1

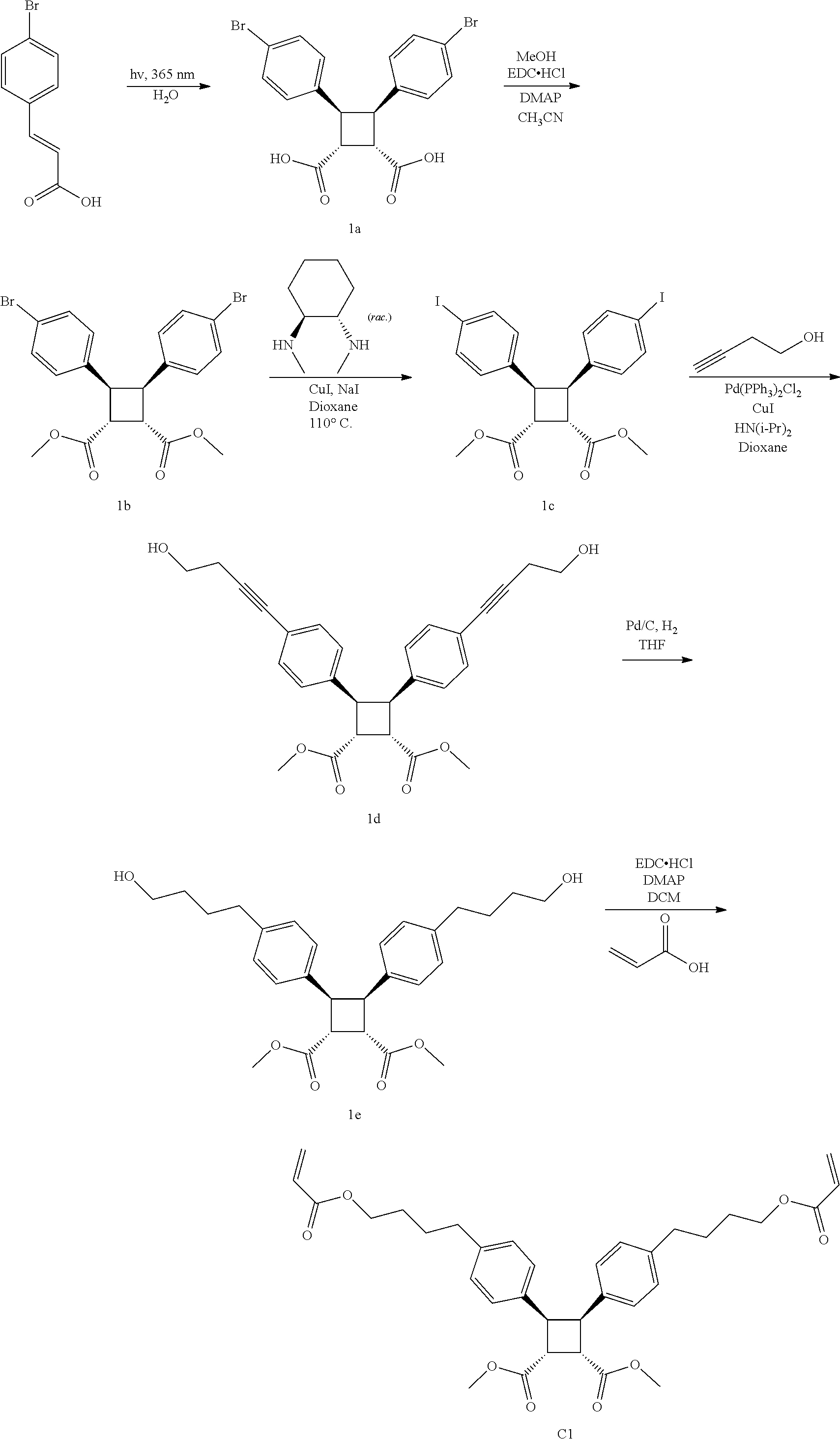

Synthesis of 1b. Compound 1a was synthesized as previously reported (Wang et al. J. Am. Chem. Soc. 143, 3714-3718 (2021)). To a 250 mL round bottom flask (RBF), compound 1a (7 g, 15.4 mmol) was mixed with 150 mL acetonitrile. N-(3-dimethylaminopropyl)-N′-ethylcarbodiimide hydrochloride (EDC-HCl) (8.9 g, 46.4 mmol) was added portion-wise. The solid first dissolved, and then the solution became cloudy again. 4-dimethylaminopyridine (DMAP) (750 mg, 6 mmol) and MeOH (2.5 mL, 62 mmol) were then added to the solution. The reaction was stirred at room temperature (r.t.) overnight. After the reaction completed, the solution was concentrated using a rotary evaporator and diluted with 200 mL ethyl acetate. The solution was washed with deionized (DI) water (150 mL×2) and brine (150 mL×1). EA phase was collected and dried with MgSO₄. After filtration, the solution was concentrated onto silica. Column chromatography (SiO₂, 0˜ 40% ethyl acetate (EtOAC)/hexane gradient eluent) gave compound 1b as a white solid (6.9 g). ¹H NMR (500 MHz, CDCl₃): δ 7.27-7.26 (d, J=8.4 Hz, 4H), 6.79-6.77 (d, J=8.4 Hz, 4H), 4.34-4.33 (m, 2H), 3.76-3.75 (m, 8H). ¹³C NMR (126 MHz, CDCl₃) δ 172.62, 138.21, 133.21, 129.55, 120.77, 54.05, 44.41, 42.79. HRMS-ESI (m/z): [M+H]⁺ calculated for C₂₀H₁₈Br₂O₄, 480.9647; observed 480.9643.

Synthesis of 1c. To a 300 mL pressure vessel charged with a stir bar, compound 1b (6.9 g, 14.3 mmol) was mixed with 35 mL dioxane. CuI (273 mg, 1.43 mmol) and NaI (8.63 g, 57.2 mmol) were added. The mixture was purged with N₂ for 10 mins. Trans-N,N-dimethylcyclohexane-1,2-diamine (448.5 μL, 2.86 mmol) was added using micropipette. The vessel was capped with PTFE cap and heated to 110° C. The reaction was stirred for 24 h. The vessel was cooled to room temperature and the mixture was poured onto a short silica plug (ethyl acetate as eluent) to give compound 1c as thick pale-yellow oil (˜8.5 g). NMR spectrum showed clean product and the compound was directly used in next step without further purification. ¹H NMR (500 MHz, CDCl₃): δ 7.47-7.46 (d, J=8.4 Hz, 4H), 6.67-6.65 (d, J=8.3 Hz, 4H), 4.32-4.21 (m, 2H), 3.74-3.73 (m, 8H). ¹³C NMR (126 MHz, CDCl₃) δ 173.84, 138.08, 137.48, 128.77, 91.38, 52.44, 45.11, 42.43.

Synthesis of 1d. To a 500 mL RBF charged with a stir bar, compound 1c (˜8.5 g, ˜ 14.3 mmol) was mixed with 150 mL Dioxane. Diisopropyl amine (12 mL, 85.8 mmol) was added. The mixture was purged with N₂ for 10 mins. Under nitrogen atmosphere, CuI (545 mg, 2.86 mmol, 0.2 equiv. to 1c) and Pd(PPh₃)₂Cl₂ (1 g, 1.43 mmol, 0.1 equiv. to 1c) were added sequentially. The solution turned dark immediately when Pd(PPh₃)₂Cl₂ was added. The reaction was stirred under N₂ atmosphere overnight. After the reaction was finished, the solution was filtered with celite. The filtrate was diluted with EA and washed with dilute (1˜2%) HCl DI water solution (150 mL×2) and brine (150 mL×1). EA phase was collected and dried over MgSO₄. Column chromatography (SiO₂, 0˜ 80% EtOAC/hexane gradient eluent) gave compound 1d as thick yellow oil (6.1 g). The yellow color was likely due to the metal residual, which can be removed by QuadraPure® TU. If not pure by NMR, repeat the chromatography. ¹H NMR (500 MHz, DMSO): δ 7.11-7.09 (d, J=8.4 Hz, 4H), 7.01-6.99 (d, J=8.5 Hz, 4H), 4.86-4.84 (t, J=5.6 Hz, 2H), 4.25-4.24 (m, 2H), 4.00-3.98 (m, 2H), 3.64 (s, 6H), 3.55-3.51 (td, J=6.9, 5.6 Hz, 4H), 2.49-2.47 (t, 4H). ¹³C NMR (126 MHz, DMSO) δ 172.04, 139.85, 129.97, 128.57, 120.37, 88.42, 81.09, 60.66, 52.46, 45.15, 42.36, 23.78. HRMS-ESI (m/z): [M+H]⁺ calculated for C₂₈H₂₈O₆, 461.1959; observed 461.1958.

Synthesis of 1e. To a 500 mL three-neck RBF charged with a stir bar, compound 1d (6.1 g, 13.2 mmol) was dissolved in 300 mL tetrahydrofuran (THF). The solution was purged with N₂ for 10 min. Under nitrogen atmosphere, Pd/C (10% Pd) (2 g) was added portion-wise. A large amount of Pd/C was added because some ligands that carried over from the last step can poison Pd/C. The mixture was purged with N₂ for another 10 min. The hydrogen balloon was connected to the RBF. The mixture was purged with hydrogen for 10 min and was allowed to react under hydrogen atmosphere for 24 h. After the reaction was finished, the solution was filtered with celite. The filtrate was loaded onto silica by rotary-evaporating the solvent. Column chromatography (SiO₂, 0˜80% EtOAC/hexane gradient eluent) gave compound 1e as thick colorless oil (3.5 g). ¹H NMR (500 MHz, CDCl₃): δ 6.91-6.90 (d, J=8.2 Hz, 4H), 6.82-6.80 (d, J=8.1 Hz, 4H), 4.34-4.32 (m, 2H), 3.82-3.81 (m, 2H), 3.74 (s, 6H), 3.60-3.58 (t, J=6.4 Hz, 4H), 2.51-2.48 (t, J=7.5 Hz, 4H), 1.60-1.45 (m, 12H). ¹³C NMR (126 MHz, CDCl₃) δ 173.76, 143.51, 136.07, 128.14, 127.08, 65.66, 53.54, 46.61, 43.37, 35.83, 33.00, 29.10. HRMS-ESI (m/z): [M+H]⁺ calculated for C₂₈H₃₆O₆, 469.2585; observed 469.2590. [M+Na]⁺ calculated for C₂₈H₃₆O₆ 491.2404; observed 491.2406.

Synthesis of C1. To a 100 mL round bottom flask (RBF), acrylic acid (461 mg, 6.4 mmol), compound 1e (1 g, 2.13 mmol), DMAP (104.3 mg, 0.85 mmol) were dissolved in 20 mL DCM. EDC-HCl (1.23 g, 6.4 mmol) was added portion-wise at the end. The reaction was stirred at r.t. overnight. After the reaction completed, the solution was concentrated using rotary evaporator and diluted with 200 mL ethyl acetate. The solution was washed with DI water (150 mL×2) and brine (150 mL×1). EA phase was collected and dried with MgSO₄. After filtration, the solution was concentrated onto silica. Column chromatography (SiO₂, 0˜40% EtOAC/hexane gradient eluent) gave compound C1 (0.75 g). Crosslinker C1 can self-crosslink at high concentration under vacuum, thus it was directly prepared as a stock solution (200˜300 mg/mL) in dioxane. ¹H NMR (500 MHz, CDCl₃): δ 6.91-6.89 (d, J=8.1 Hz, 4H), 6.82-6.81 (d, J=8.1 Hz, 4H), 6.40-6.36 (dd, J=17.3, 1.5 Hz, 2H), 6.13-6.07 (dd, J=17.3, 10.4 Hz, 2H), 5.82-5.80 (dd, J=10.5, 1.5 Hz, 2H), 4.34-4.33 (m, 2H), 4.13-4.10 (t, 4H), 3.83-3.80 (m, 2H), 3.74 (s, 6H), 2.52-2.48 (t, 4H), 1.62-1.57 (p, 8H). ¹³C NMR (126 MHz, CDCl₃) δ 172.44, 168.45, 140.09, 136.21, 131.07, 128.69, 128.14, 127.94, 65.19, 52.27, 44.80, 42.96, 35.75, 28.12, 27.64. HRMS-ESI (m/z): [M+H]⁺ calculated for C₃₄H₄₀O₈, 577.2796; observed 577.2798. [M+Na]⁺ calculated for C₃₄H₄₀O₈ 599.2615; observed 599.2612.

### Synthesis of C2

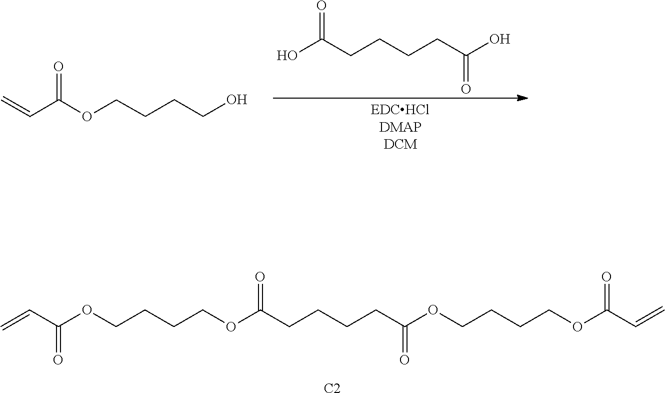

To a 500 mL RBF, adipic acid (4 g, 27.3 mmol), EDC-HCl (10.5 g, 55 mmol), DMAP (0.67 g, 5.5 mmol) were added sequentially to 200 mL dichloromethane (DCM). 4-hydroxybutyl acrylate (10 g, 111 mmol) was added at the end. The reaction was stirred at r.t. for overnight. After the reaction completed, the solution was concentrated using a rotary evaporator and diluted with 200 mL ethyl acetate. The solution was washed with DI water (150 mL×2) and brine (150 mL×1). EA phase was collected and dried with MgSO₄. After filtration, the solution was concentrated onto silica. Column chromatography (SiO₂, 0˜40% EtOAC/hexane gradient eluent) gave compound C2 (˜6 g). Crosslinker C2 can self-crosslink at high concentration under vacuum, thus it was directly prepared as a stock solution (200˜300 mg/mL) in dioxane. ¹H NMR (500 MHz, CDCl₃): δ 6.34-6.40 (dd, J=17.4, 1.4 Hz, 2H), 6.13-6.07 (dd, J=17.3, 10.4 Hz, 2H), 5.83-5.80 (dd, J=10.4, 1.5 Hz, 2H), 4.18-4.18 (t, J=6.0 Hz, 4H), 4.08-4.10 (t, J=6.1 Hz, 4H), 2.33-2.30 (m, 4H), 1.76-1.63 (m, 12H). ¹³C NMR (126 MHz, CDCl₃) δ 173.41, 166.29, 130.85, 128.55, 64.10, 63.95, 33.96, 25.42, 24.48. HRMS-ESI (m/z): [M+H]⁺ calculated for C₂₀H₃₀O₈, 399.2013; observed 399.2013. [M+Na]⁺ calculated for C₂₀H₃₀O₈ 421.1833; observed 421.1836

### Synthesis of C3

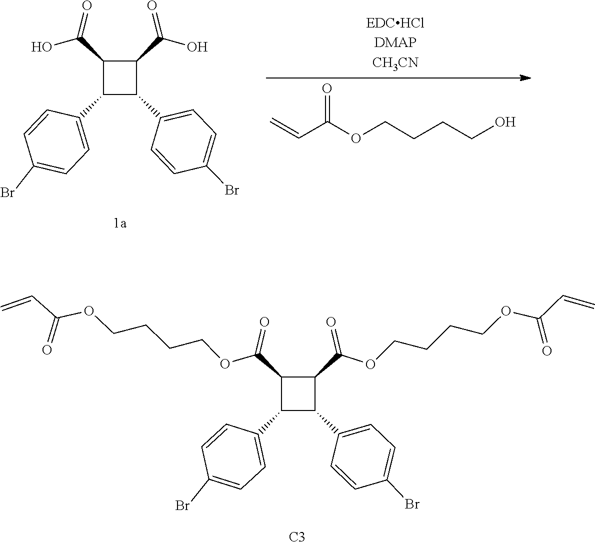

To a 500 mL round bottom flask (RBF), 1a (2 g, 4.4 mmol), EDC-HCl (1.69 g, 8.9 mmol), DMAP (108 mg, 0.88 mmol) were added sequentially to 50 mL acetonitrile. 4-hydroxybutyl acrylate (1.4 g, 9.7 mmol) was added at the end. The reaction was stirred at r.t. overnight. After the reaction completed, the solution was concentrated using a rotary evaporator and diluted with 200 mL ethyl acetate. The solution was washed with DI water (150 mL×2) and brine (150 mL×1). EA phase was collected and dried with MgSO₄. After filtration, the solution was concentrated onto silica. Column chromatography (SiO₂, 0˜40% EtOAC/hexane gradient eluent) gave compound C3 (˜2 g). Crosslinker C3 can self-crosslink at high concentration under vacuum, thus it was directly prepared as a stock solution (200˜300 mg/mL) in dioxane. ¹H NMR (500 MHz, CDCl₃): δ 7.28-7.26 (d, J=8.3 Hz, 4H), 6.81-6.79 (d, J=8.2 Hz, 4H), 6.42-6.38 (dd, J=17.4, 1.4 Hz, 2H), 6.15-6.09 (dd, J=17.3, 10.4 Hz, 2H), 5.84-5.82 (dd, J=10.4, 1.4 Hz, 2H), 4.33-4.32 (m, 2H), 4.20-4.17 (m, 8H), 3.76-3.74 (m, 2H), 1.78-1.71 (m, 8H). ¹³C NMR (126 MHz, CDCl₃) δ172.16, 166.26, 137.38, 131.47, 130.93, 129.54, 128.50, 120.74, 64.81, 64.00, 44.50, 43.41, 25.38, 25.36. HRMS-ESI (m/z): [M+H]⁺ calculated for C₃₂H₃₄Br₂O₈, 705.0693; observed 705.0681. [M+NH₄]⁺ calculated for C₃₂H₃₄Br₂O₈ 722.0959; observed 722.0950. [M+Na]⁺ calculated for C₃₂H₃₄Br₂O₈ 727.0513; observed 727.0539.

### Synthesis of C4

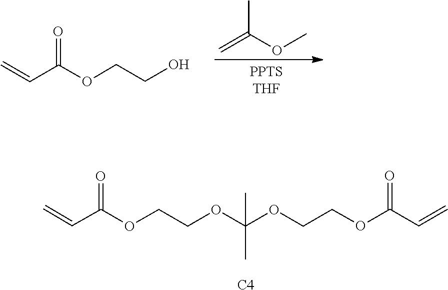

To a 50 mL round bottom flask (RBF) charged with a stir bar, 2-hydroxyethyl acrylate (5 g, 4.95 mL, 43 mmol) and pyridinium p-toluenesulfonate (PPTS) (500 mg, 2 mmol) were added sequentially to 15 mL THF. 2-Methoxypropene (1.67 g, 2.22 mL, 23 mmol) solution in 15 mL THE was added dropwise at 0° C. The reaction was allowed to react overnight. After the reaction was finished, a few drops of TEA were added to the solution, and the solution was concentrated onto silica. Column chromatography (SiO₂, 0˜ 40% EtOAC/hexane gradient eluent, 1% TEA was added) gave compound C4 (˜3 g). Crosslinker C4 can self-crosslink at high concentration under vacuum, thus it was directly prepared as a stock solution (˜200 mg/mL) in DCM. ¹H NMR (500 MHz, CDCl₃): δ 6.40-6.36 (dd, J=17.3, 1.5 Hz, 2H), 6.13-6.08 (dd, J=17.4, 10.4 Hz, 2H), 5.82-5.77 (dd, J=10.5, 1.5 Hz, 2H), 4.29-4.19 (m, 4H), 3.67-3.65 (m, 4H), 1.35 (s, 6H). ¹³C NMR (126 MHz, CDCl₃) δ 166.19, 131.03, 128.37, 100.29, 63.93, 58.97, 24.84. HRMS-ESI (m/z): [M+Na]⁺ calculated for C₁₃H₂₀O₆ 295.1152; observed 295.1152.

### Synthesis of P1

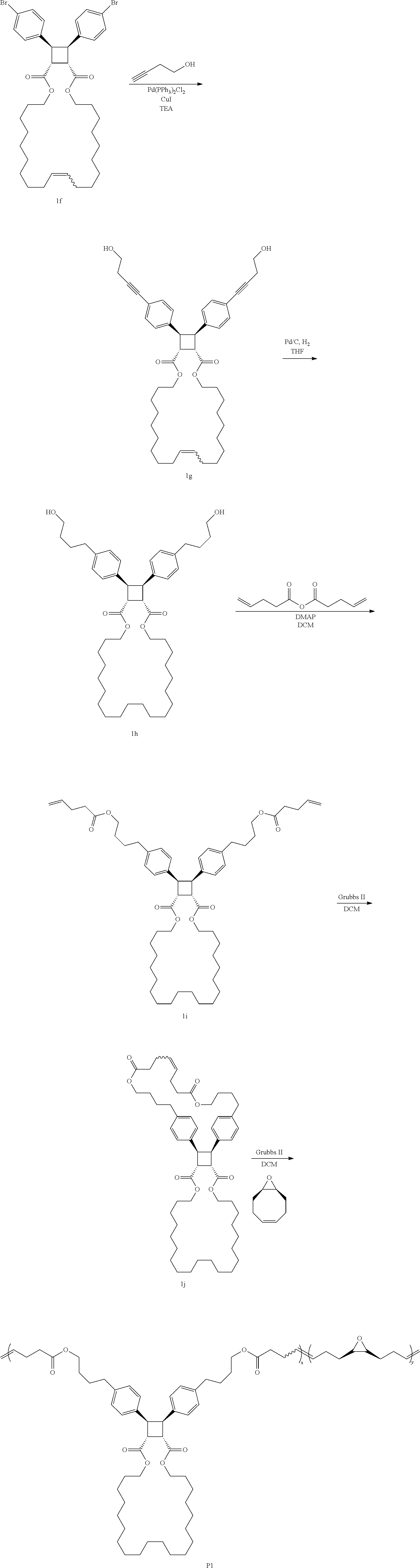

Synthesis of 1g. Compound if was synthesized as previously reported (Bowser 2021). To a 25 mL RBF charged with a stir bar, compound if (500 mg, 0.68 mmol) and 3-Butyn-lol (124 μL, 115 mg, 1.6 mmol) were mixed with 8.2 mL TEA. The mixture was purged with N₂ for 10 mins. Under nitrogen atmosphere, CuI (2.6 mg, 13.6 μmol, 0.02 equiv. to 10) and Pd(PPh₃)₂Cl₂ (19.2 mg, 27.4 μmol, 0.04 equiv. to if) were added sequentially. The reaction was heated to 60° C. and stirred under N₂ atmosphere for overnight. After the reaction was finished, TEA was evaporated using rotary evaporator. The mixture was diluted with EA and washed with dilute (1˜2%) HCl DI water solution (150 mL×2) and brine (150 mL×1). EA phase was collected and dried over MgSO₄. Column chromatography (SiO₂, 0˜80% EtOAC/hexane gradient eluent) gave compound 1g as thick yellow oil (347 mg). The yellow color was likely due to the metal residual, which can be removed by QuadraPure® TU. ¹H NMR (500 MHz, CDCl₃): δ 7.17-7.15 (d, J=8.2 Hz, 4H), 6.85-6.83 (d, J=8.0 Hz, 4H), 5.35-5.31 (m, 2H), 4.37-4.36 (m, 2H), 4.16-4.05 (m, 4H), 3.80-3.74 (m, 6H), 2.66-2.63 (t, J=6.2 Hz, 4H), 2.06-2.00 (m, 4H), 1.66-1.60 (m, 4H), 1.36-1.26 (m, 24H). ¹³C NMR (126 MHz, CDCl₃) δ 172.32, 138.70, 131.58, 130.97, 127.85, 121.48, 86.48, 65.64, 61.27, 44.82, 43.48, 32.19, 29.77, 29.54, 29.44, 29.00, 28.77, 28.19, 26.41, 23.96. HRMS-ESI (m/z): [M+H]⁺ calculated for C₄₆H₆₀O₆, 709.4463; observed 709.4467.

Synthesis of 1h. To a 50 mL three-neck RBF charged with a stir bar, compound 1g (347 mg, 0.49 mmol) was dissolved in 25 mL THF. The solution was purged with N₂ for 10 min. Under nitrogen atmosphere, Pd/C (10% Pd) (30 mg) was added portion-wise. The mixture was purged with N₂ for another 10 min. The hydrogen balloon was connected to the RBF. The mixture was purged with hydrogen for 10 seconds and was allowed to react under hydrogen atmosphere for 48 hours. After the reaction was finished, the solution was filtered with celite. The filtrate was loaded onto silica by rotary-evaporating the solvent. Column chromatography (SiO₂, 0˜80% EtOAC/hexane gradient eluent) gave compound 1h as thick colorless oil (312 mg). ¹H NMR (500 MHz, CDCl₃): δ 6.91-6.89 (d, J=7.9 Hz, 4H), 6.82-6.81 (d, J=8.0 Hz, 4H), 4.34-4.33 (m, 2H), 4.15-4.06 (m, 4H), 3.80-3.78 (m, 2H), 3.60-3.57 (t, J=6.4 Hz, 4H), 2.51-2.48 (t, J=7.4 Hz, 4H), 1.65-1.46 (m, 16H), 1.37-1.28 (m, 30H). ¹³C NMR (126 MHz, CDCl₃) δ 172.71, 140.29, 136.37, 128.09, 127.92, 65.32, 62.84, 44.72, 43.63, 35.16, 32.22, 29.21, 29.16, 28.84, 28.72, 28.51, 28.13, 27.65, 27.63, 27.48, 25.90. HRMS-ESI (m/z): [M+H]⁺ calculated for C₄₆H₇₀O₆, 719.5245; observed 719.5249.

Synthesis of 1i. To a 25 mL RBF charged with a stir bar, compound 1h (312 mg, 0.43 mmol) and DMAP (10 mg, 82.9 μmol) were dissolved in 10 mL DCM. 4-pentenoic anhydride (166.5 μL, 166 mg, 0.91 mmol, 2.1 equiv. to 1 h) was added dropwise. The reaction was stirred overnight. After the reaction was finished, the solution was loaded onto silica by evaporating solvent. Column chromatography (SiO₂, 0˜20% EtOAc/hexane gradient eluent) gave compound 1i as colorless oil (291 mg). ¹H NMR (500 MHz, CDCl₃): δ 6.90-6.89 (d, J=7.9 Hz, 4H), 6.83-6.81 (d, J=7.9 Hz, 4H), 5.77-5.85 (ddt, J=16.4, 10.2, 6.0 Hz, 2H), 5.06-4.79 (m, 4H), 4.33-4.34 (m, 2H), 4.15-4.06 (qt, J=10.8, 7.0 Hz, 4H), 4.05-4.03 (m, 4H), 3.79-3.77 (m, 2H), 2.50-2.48 (m, 4H), 2.41-2.33 (m, 8H), 1.67-1.61 (p, J=7.0 Hz, 4H), 1.57-1.53 (m, 8H), 1.37-1.30 (m, 32H). ¹³C NMR (126 MHz, CDCl₃) δ 173.24, 172.67, 139.98, 136.84, 136.50, 128.08, 127.97, 115.61, 65.33, 64.34, 44.67, 43.75, 34.96, 33.69, 29.20, 29.16, 29.02, 28.84, 28.72, 28.51, 28.17, 28.13, 27.65, 27.62, 25.90. HRMS-ESI (m/z): [M+H]⁺ calculated for C₅₆H₈₂O₈, 883.6083; observed 883.6078. [M+NH₄]⁺ calculated for C₅₆H₈₂O₈ 900.6348; observed 900.6342. [M+Na]⁺ calculated for C₅₆H₈₂O₈ 905.5902; observed 905.5900.

Synthesis of 1j. To a 500 mL RBF charged with a stir bar, compound 1i (291 mg, 0.33 mmol) was dissolved in 200 mL DCM. Grubbs 2^{nd} catalyst (14 mg, 16.5 μmol, 0.05 equiv. to 1i) was then added. The solution was refluxed overnight and quenched with several drops of vinyl ethyl ether. The solution was loaded onto silica gel by evaporating the solvent. Column chromatography (SiO₂, 0˜20% EtOAc/hexane gradient eluent) gave compound 1j as colorless oil (220 mg). ¹H NMR (500 MHz, CDCl₃): δ 6.88-6.85 (m, 4H), 6.80-6.77 (m, 4H), 5.46-5.39 (m, 2H), 4.33-4.32 (m, 2H), 4.16-4.07 (m, 4H), 4.03-4.00 (m, 4H), 3.82-3.80 (m, 2H), 2.50-2.47 (t, J=7.2 Hz, 4H), 2.35-2.30 (m, 8H), 1.67-1.62 (p, J=7.0 Hz, 4H), 1.57-1.45 (m, 8H), 1.37-1.30 (m, 32H). ¹³C NMR (126 MHz, CDCl₃) δ 173.19, 172.70, 139.86, 136.30, 129.64, 127.98, 65.34, 64.27, 44.88, 43.19, 34.81, 34.30, 29.21, 29.17, 28.85, 28.73, 28.53, 28.14, 28.01, 27.95, 27.67, 27.64, 27.56, 25.91. HRMS-ESI (m/z): [M+H]⁺ calculated for C₅₄H₇₈O₈, 855.5770; observed 855.5777.

Synthesis of P1. A 2 mL crimp top vial was charged with 1j (46.3 mg, 0.4 equiv.) and freshly distilled 9-oxabicyclo[6.1.0]non-4-ene (10.4 mg, 0.6 equiv.) under N₂ (g). A stock solution of Grubbs Catalyst 2nd Generation in dry DCM (0.66 mg/mL) was prepared and sparged with N₂ (g) for 10 minutes. Then, the stock solution (0.1 mL) containing the Grubbs Catalyst (1/1500 equiv.) was added via an air-tight syringe to dissolve the monomers to the concentration of 1 M and initiate the polymerization. After 16 hours, the polymerization was quenched with 5 drops of ethyl vinyl ether and then precipitated into methanol to give the crude polymer. Polymers were purified via two additional precipitations into MeOH and one reverse precipitation from DCM. The polymer was dried on a high vacuum for at least 1 hour prior to use. ¹H NMR (500 MHz, CDCl₃): δ 6.90-6.88 (m, 4H), 6.83-6.81 (m, 4H), 5.51-5.44 (m, 4.7H), 4.34-4.33 (m, 2H), 4.15-4.10 (m, 8H), 3.78-3.77 (m, 2H), 2.94-2.88 (m, 2.9H), 2.50-2.47 (m, 4.3H), 2.36-2.12 (m, 13.7H), 1.67-1.54 (m, 20H), 1.37-1.29 (m, 33H). Mₙ=354 kDa, PDI=1.38.

### Synthesis of C5

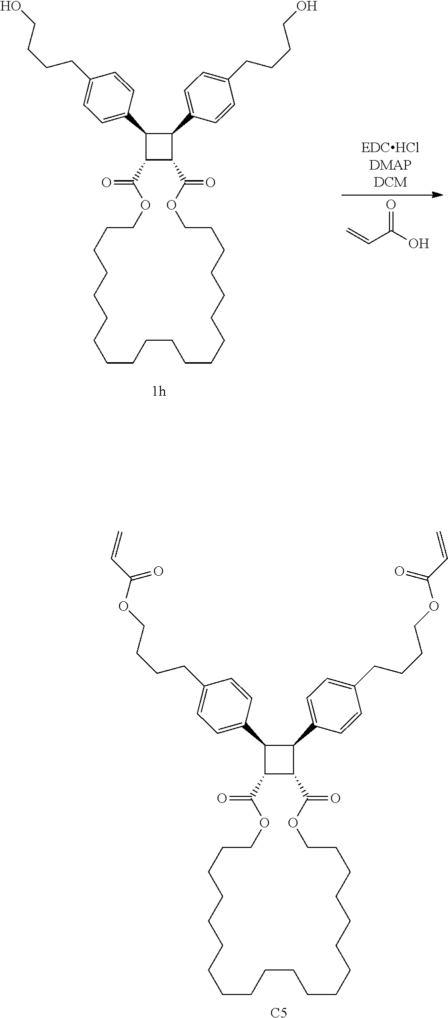

To a 100 mL round bottom flask (RBF), acrylic acid (240 mg, 3.3 mmol), compound 1h (800 mg, 1.11 mmol), DMAP (54.3 mg, 0.44 mmol) were dissolved in 20 mL DCM. EDC-HCl (640 mg, 6.4 mmol) was added portion-wise at the end. The reaction was stirred at r.t. overnight. After the reaction was completed, the solution was concentrated onto silica. Column chromatography (SiO₂, 0˜ 40% EtOAC/hexane gradient eluent) gave compound C5 (340 mg). Crosslinker C5 can self-crosslink at high concentration under vacuum, thus it was directly prepared as a stock solution (200˜ 300 mg/mL) in DCM. ¹H NMR (500 MHz, CDCl₃): δ 6.91-6.89 (d, J=8.1 Hz, 4H), 6.83-6.81 (d, J=8.1 Hz, 4H), 6.40-6.36 (dd, J=17.3, 1.5 Hz, 2H), 6.13-6.07 (dd, J=17.3, 10.4 Hz, 2H), 5.82-5.79 (dd, J=10.5, 1.5 Hz, 2H), 4.34-4.33 (m, 2H), 4.16-4.06 (m, 8H), 3.79-3.78 (m, 2H), 2.52-2.49 (t, 4H), 1.66-1.58 (m, 12H), 1.34-1.30 (m, 32H). ¹³C NMR (126 MHz, CDCl₃) δ 172.65, 166.39, 139.95, 136.48, 130.67, 128.68, 128.08, 127.97, 65.32, 64.50, 44.68, 43.72, 34.95, 29.20, 29.16, 28.84, 28.73, 28.51, 28.13, 28.12, 27.65, 27.63, 25.90. HRMS-ESI (m/z): [M+H]⁺ calculated for C₅₂H₇₄O₈, 827.5457; observed 827.5456. [M+NH₄]⁺ calculated for C₅₂H₇₄O₈ 844.5722; observed 844.5719. [M+Na]⁺ calculated for C₅₂H₇₄O₈ 849.5276; observed 849.5277.

### Synthesis of 1k

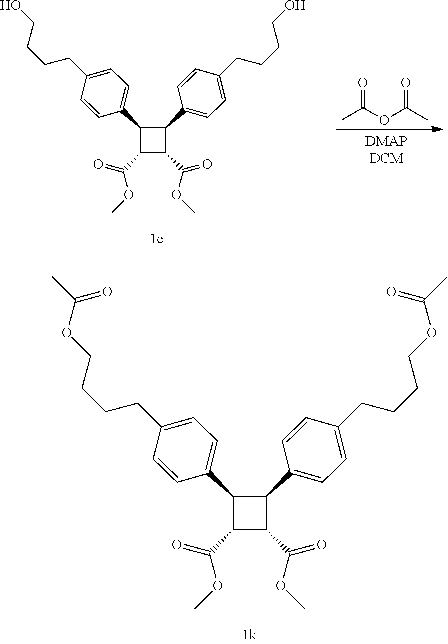

To a 25 mL RBF charged with a stir bar, compound 1e (200 mg, 0.43 mmol) and DMAP (31 mg, 256 μmol) were dissolved in 10 mL DCM. Acetic anhydride (101 μL, 109 mg, 1.07 mmol, 2.5 equiv. to 1e) was added dropwise. The reaction was stirred overnight. After the reaction was finished, the solution was loaded onto silica by evaporating solvent. Column chromatography (SiO₂, 0˜40% EtOAc/hexane gradient eluent) gave compound 1k as colorless oil (101 mg). ¹H NMR (500 MHz, CDCl₃): δ 6.91-6.89 (d, J=8.0 Hz, 4H), 6.82-6.81 (d, J=8.2 Hz, 4H), 4.34-4.33 (m, 2H), 4.03-4.01 (m, 4H), 3.81-3.80 (m, 2H), 3.74 (s, 6H), 2.51-2.48 (m, 4H), 2.03 (s, 6H), 1.56-1.53 (m, 8H). ¹³C NMR (126 MHz, CDCl₃) δ 173.16, 171.31, 140.10, 136.21, 128.14, 127.94, 64.45, 52.28, 44.80, 43.50, 34.98, 28.14, 27.66, 21.14. HRMS-ESI (m/z): [M+H]⁺ calculated for C₃₂H₄₀O₈, 553.2796; observed 553.2796. [M+Na]⁺ calculated for C₃₂H₄₀O₈ 575.2614; observed 575.2614.

### Example 2—Network Preparation and Characterization Methods

Network Preparation

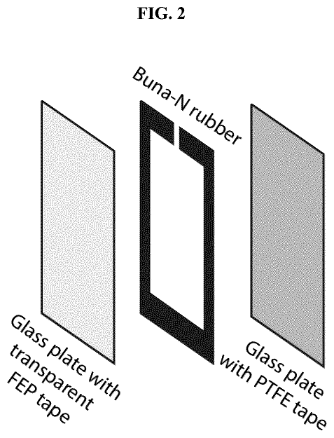

Fresh acrylate monomer was prepared by passing it through a basic aluminum oxide pad to remove the inhibitor before every network preparation. Here we use a pair of elastomers (E1 and E2) with a stoichiometry of [M]:[C]:[CTA]:[PI]=1:1/50:1/1200:1/2000 as an example. Other elastomers were prepared in the same manner. A pre-gel solution was prepared by mixing 2-methoxyethyl acrylate (M) (7 g, 1 equiv.), 4-Cyano-4-(dodecylsulfanylthiocarbonyl)sulfanyl]pentanoic acid (CTA) (18.1 mg, 1/1200 equiv.) and α-ketoglutaric acid (PI) (3.93 mg, 1/2000 equiv.) in a 45 mL scintillation vial. The pre-gel solution was sonicated for 5 minutes to obtain a homogeneous solution. The pre-gel solution was separately transferred into two 20 mL scintillation vials (3 mL each) with a pipette. The desired amount of different stock solutions of crosslinkers (200˜300 mg/mL) was separately added to these two vials to obtain two solutions with the same ratio of [M]: [C]: [CTA]: [PI]=1:1/50:1/1200:1/2000. Since the stock solution of crosslinkers may have had different concentrations, the desired amount of solvent was added to ensure the same concentration for both solutions. These two vials with different crosslinkers were put onto a rotary evaporator to remove the volatile component. Afterward, the solutions were purged with nitrogen for 3 minutes. Each solution was passed through a 1 m pore size PTFE syringe filter to remove dust and transferred into a “sandwich” mold (FIG. 2) using syringes. The molds were placed in a nitrogen-purged glove bag and were irradiated with 365 nm UV light (Analytik Jena, UVL-28 UV lamp, 8 Watt) for at least 20 hours to ensure good crosslinking. After crosslinking, the network films were taken out and blown with nitrogen overnight to remove solvent residue. The dried films were cut into desired shapes for the below listed mechanical tests.

Network Characterization

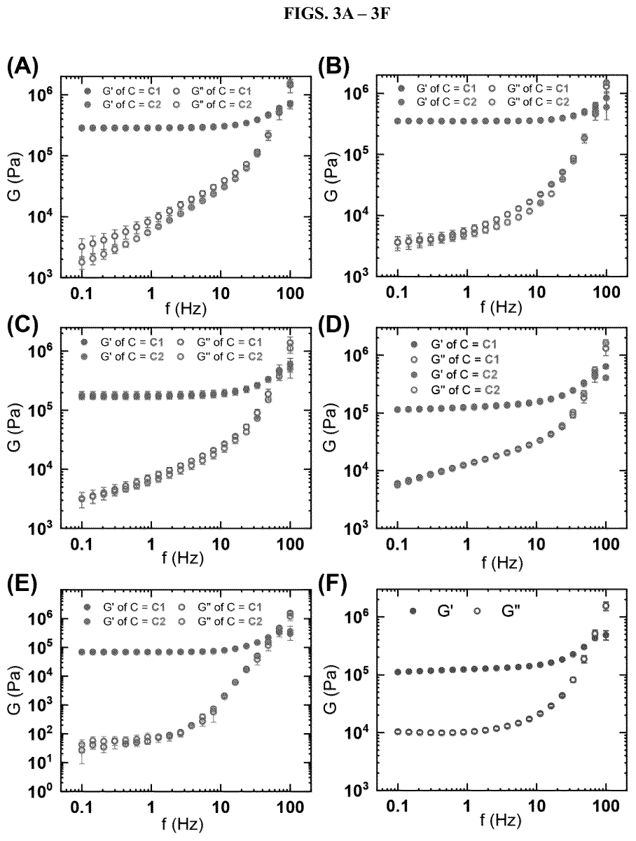

Rheology. Network films were cut to size with an 8 mm diameter biopsy punch. Rheological measurements were conducted on an Anton Paar MCR 302 rheometer with an 8 mm parallel plate geometry. Frequency sweep measurements were conducted at 22-23° C. with a constant 0.5% shear strain, well within the linear viscoelastic regime based on initial strain sweeps. Constant temperature was maintained with a Peltier temperature control stage. Three samples from different positions in the network film were punched out for shear moduli measurement. Frequency sweep data are shown in FIGS. 3A-3F.

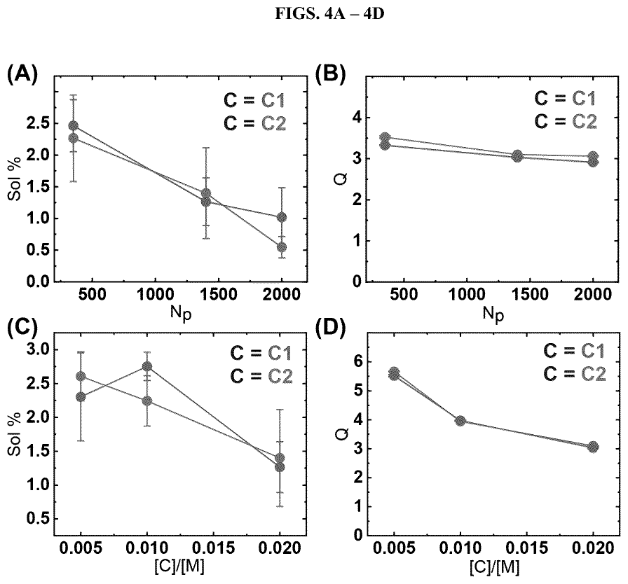

Sol fraction and equilibrium swelling ratio. After rheology tests, the disc samples (×3) were weighted and submerged in ethyl acetate solvent to extract sol fractions. The solvent was changed every 24 hours until the mass of the dry samples stops changing. The mass of the dry samples usually reaches constant after three times of solvent exchange. The sol fractions were calculated by (mass loss)/(initial mass)×100%. The dry samples were then submerged in DMF to reach equilibrium swelling. The equilibrium swelling ratios were calculated by (mass of the swollen sample)/(mass of the dry sample). The results of sol fraction and equilibrium swelling ratios are shown in FIGS. 4A-4D.

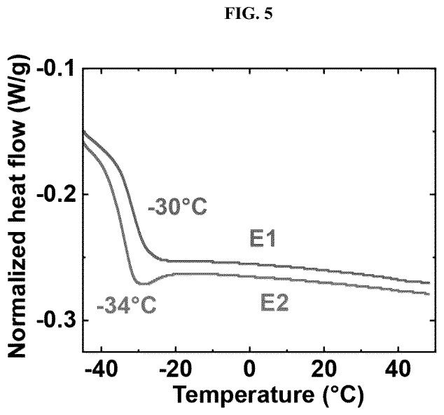

Differential Scanning Calorimetry (DSC). Samples of E1 and E2 were performed following this procedure (−50° C. to 50° C., 10° C./min; isothermal 50° C., 3 min; 50 to −50° C., 10° C./min; isothermal −50° C., 3 min; −50° C. to 50° C., 10° C./min; isothermal 50° C., 3 min; 50 to −50° C., 10° C./min), wherein the thermal transitions for the heating cycle were recorded. The glass transition temperature (T_{g}) was determined by the inflection point of the heat capacity with temperature sweep (second cycle). The T_{g} of E2 is consistent with reported data (Chen et al. J. Mater. Chem. A 7, 17334-17344 (2019)) of linear poly 2-methoxyethyl acrylate, where T_{g} of E1 is slightly higher, but still well below room temperature (FIG. 5).

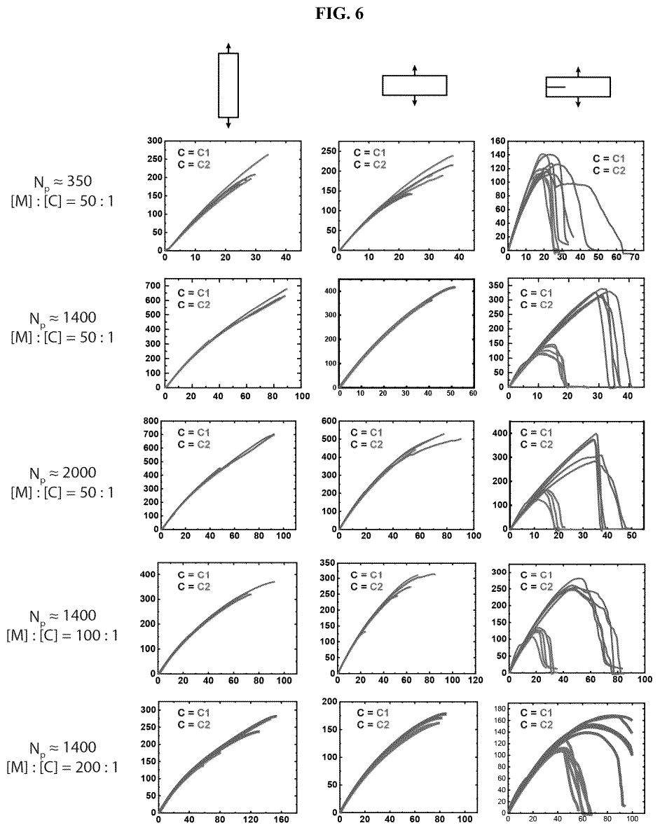

Tearing energy and fatigue threshold. The fatigue tests were conducted following the previous method (Wang et al. J. Am. Chem. Soc. 143, 3714-3718 (2021)). Samples for tearing energy were cut with a razor blade as a 15×20 mm rectangle. This was reduced to a 20×3 mm area after clamping. Typically, each measurement used 3 un-notched samples and 5 notched samples; for notched samples, a ˜5 mm cut was made in the center of one side of the piece, perpendicular to the edge. The exact thickness and width were measured with calipers before each test. Samples were loaded into the clamps at a gauge length of ˜2 mm, then stretched to a force of about 0.01 N, which resulted in an initial gauge length of 2.5+0.5 mm. Unnotched samples and notched samples were pulled to failure at a constant strain rate of 0.2% s⁻¹. Tearing energy was calculated using the Rivlin-Thomas method (Rivlin et al. J. Polym. Sci. 10, 291-318 (1953)) where the strain energy is obtained by integrating the un-notched stress-strain curve to the strain at which the crack of the notched samples began to propagate. Results are shown in FIG. 6 and Table 1.

TABLE 1
Summary of tearing energies for elastomers in FIG. 6. In each cell, the top
and bottom entries indicate the tearing energies for elastomer made from
C1 and C2, respectively. The values are the mean ± standard deviation.

| | [M]:[C] | | | | | |
|---|---|---|---|---|---|---|
| Nₚ | 200:1 | | 100:1 | | 50:1 | |
| 350 | — | | — | | 21.9 ± 4.9 | J · m⁻² |
| | | | | | 15.6 ± 5.6 | J · m⁻² |
| 1400 | 248.3 ± 16.6 | J · m⁻² | 148.6 ± 3.2 | J · m⁻² | 113.0 ± 9.7 | J · m⁻² |
| | 66.7 ± 5.2 | J · m⁻² | 23.7 ± 3.1 | J · m⁻² | 11.5 ± 3.7 | J · m⁻² |

| | | | | |
|---|---|---|---|---|
| 2000 | — | — | 135.8 ± 11.6 | J · m⁻² |

| | |
|---|---|
| 19.8 ± 4.1 | J · m⁻² |

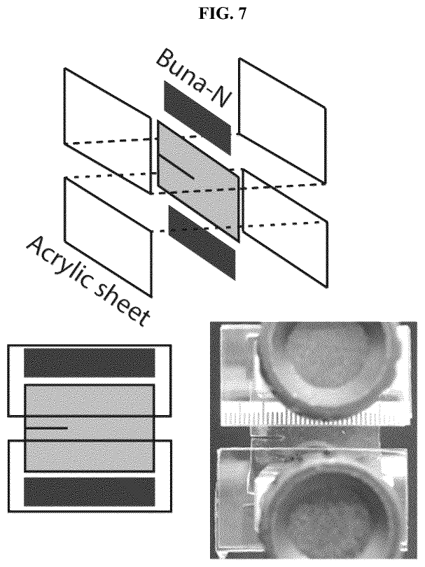

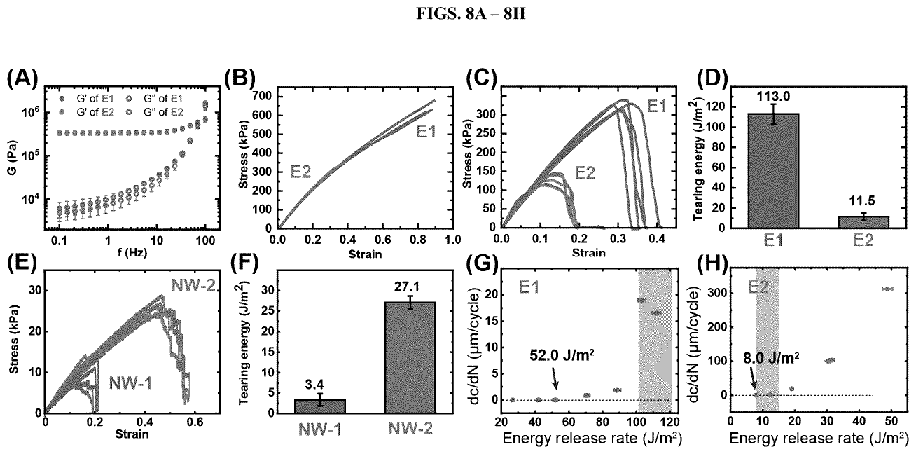

The fatigue tests were conducted based on a method described by Suo, Zhao, and coworkers (Grandbois et al. Science 283, 1727-1730 (1999); Wang et al. Science 374, 193-196 (2021)). Samples for the fatigue test were cut with a razor blade as a 30 mm×30 mm rectangle. This was reduced to around 30×3 mm area after clamping. Acrylic sheets and Buna-N rubber spacers were used to achieve a wide and firm grip for these samples (FIG. 7). A ruler tape was attached to the acrylic grip. Each measurement used 3 un-notched samples to obtain uniaxial tensile stress-strain curves. For notched samples, a ˜ 5 mm cut was made in the center of one side of the piece, perpendicular to the edge. These samples were subjected to a displacement-controlled cyclic load with a frequency of 1 Hz at a constant displacement rate. The crack propagation was monitored with a webcam (Logitech HD Pro C920), which takes a photo of the sample every second. Based on the photos, the growth of crack length c per cycle (dc/dN) can be estimated. For samples that did not have observable crack propagation, at least one thousand cycles were applied. The energy release rate was calculated by integrating the un-notched stress-strain curves to the strain applied and multiplying it by the initial height of the sample. With these data, dc/dN against the energy release rate can be plotted (FIG. 8G-8H).

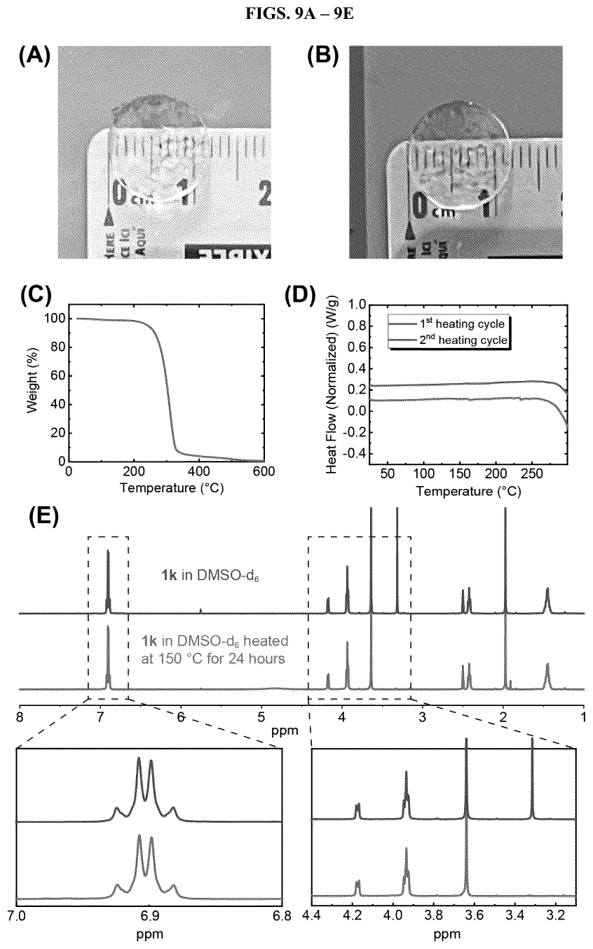

Thermal stability of E1 and its cyclobutane crosslinks. Elastomer E1 prepared with a stoichiometry [M]: [C]: [CTA]: [PI]=1:1/50:1/1200:1/2000 was submerged in DMF to reach equilibrium swelling (FIG. 9A). The fully swollen gel was submerged in DMF and heated to 100° C. for 96 hours. The swollen gel was taken out and cooled to room temperature. No further swelling was observed (FIG. 9B), indicating crosslinker C1 has good thermal stability to maintain the network integrity at 100° C. Although the thermal stability of the cyclobutane structure of C1 has been investigated by Boulatov and coworkers (Zhang et al. Nat. Commun. 8, 1147 (2017)), the characterizations of its thermal stability are still provided herein.

Compound 1k was synthesized from 1e to mimic the crosslinks in E1. Thermogravimetric analysis (TGA) (FIG. 9C) was performed on 1k following this procedure (ramp 20.00° C./min from 25° C. to 800.00° C.). DSC measurements were conducted on compound 1k (FIG. 9D) following this procedure (20° C. to 300° C., 10° C./min; isothermal 300° C., 2 min; 300 to 20° C., 10° C./min; isothermal 20° C., 2 min; 20° C. to 300° C., 10° C./min; isothermal 300° C., 2 min; 300 to 20° C., 10° C./min), wherein the thermal transitions for the heating cycles were plotted. TGA indicates the weight loss of compound 1k starts at around 260° C. It is possible that decomposition happens below 260° C. without having weight loss, however, DSC of 1k (sample mass=4.09 mg) does not show strong heat flow from room temperature to ˜260° C. in the first and the second heating cycles. The lack of strong heat flow in DSC suggests the chemical decomposition is unlikely to happen within this temperature range, unless the energy difference between 1k and the decomposed products is very small. To further confirm the thermal stability of 1k, its NMR spectra in DMSO-d₆ was investigated before (FIG. 9E, black) and after heating at 150° C. for 24 h (FIG. 9E, red). No obvious change was observed, suggesting that the cyclobutane structure is very stable even at 150° C.

### Example 3—General Characterization of Mechanophores

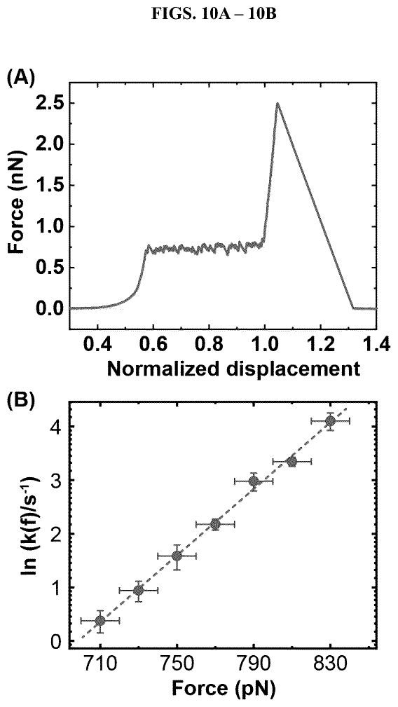

The approach is shown in FIG. 1C. Side-chain crosslinked networks with long polyacrylate backbones (primary chain, light grey chains in FIGS. 1B-1C) are formed through reversible addition-fragmentation chain transfer (RAFT) polymerization of 2-methoxyethyl acrylate monomer (M). The polymerization is initiated by the photoinitiator (PI) and the average degree of polymerization of the primary chains is controlled by the chain transfer agent (CTA). The primary chain in this system is defined as the polyacrylate backbone formed through radical polymerization. For example, polymerizing a pre-gel solution without cross-linker (C) directly yields primary chains (linear polyacrylate) as a melt with no significant mechanical integrity. Polymerizing the pre-gel solution with bis-acrylate cross-linkers C1 and C2 separately, however, forms two percolated elastomer networks E1 and E2 because the primary chains in E1 and E2 are held together by cross-linkers C1 and C2. Cross-linker C1 is a cis-diaryl substituted cyclobutane-based mechanophore which reacts under tension by means of a force-coupled [2+2]cycloreversion to form two cinnamates (FIG. 1D) (Zhang 2017), while cross-linker C2 consists of common hydrocarbon and carbon-oxygen bonds which are mechanochemically strong (Grandbois et al. Science 283, 1727-1730 (1999); Beyer et al. J. Chem. Phys. 112, 7307-7312 (2000)). The force-coupled kinetics of C1 cycloreversion have been characterized by single-molecule force spectroscopy (SMFS) (FIGS. 10A-10B). The lifetime-force relationship of C1 and common C—C bond (simulation data by Beyer (Beyer 2000)) are shown in FIG. 1E. The force needed to achieve lifetimes relevant to material tearing (between microseconds and seconds) is roughly a factor of 5 lower for C1 than what is expected for C2 and the other molecular components of the networks.

### Example 4—Form and Toughness of Mechanophores

Experiments were started with a stoichiometry of [M]: [C]: [CTA]: [PI]=1:1/50:1/1200:1/2000. The reactivity of alkyl acrylates is largely independent of the character of the alkyl group (Haehnel et al. Macromolecules 47, 3483-3496 (2014)). Thus, polymerizing the same pre-gel solution with C1 and C2 will lead to effectively identical networks that differ only in the mechanically coupled reactivity of the cross-linker. This was verified through several characterizations. First, shear moduli of E1 and E2 (blue and red in FIG. 8A, respectively) were characterized by small-amplitude oscillatory rheology and both exhibit similar storage moduli G′ and loss moduli G″ across frequencies of 0.1-100 Hz. Their storage moduli G′ are independent of frequency from 0.1-10 Hz and are well above G″, indicating E1 and E2 are well-formed elastic networks. The average G′ of E1 and E2 at 0.1 Hz are statistically the same (p=0.83, T-test assuming equal variance): 336±35 and 330±14 kPa, respectively, and well above the modulus due to entanglement Gₑ≈111±6 kPa (FIGS. 3A-3F). Second, Young's moduli obtained from uniaxial stress-strain curves (FIG. 8B) are indistinguishable. Third, similar sol fractions (FIG. 4A), and, fourth, similar equilibrium swelling ratios (FIG. 4B) demonstrate that E1 and E2 are statistically identical in terms of network connectivity. Furthermore, the glass transition temperatures T_{g} of E1 and E2 are similar and well below room temperature (˜−30° C.) (FIG. 5).

Although these two elastomers have similar network connectivity, it was noticed that their unnotched films break very differently when stretched either by hand (not shown) or by dynamic mechanical analysis (DMA) (FIG. 8B). The network E1 made with weaker cross-linkers is noticeably more difficult to tear than E2 made with stronger cross-linkers. To quantitatively confirm their difference, the tearing energies T of E1 and E2 were characterized using the Rivlin-Thomas method on notched films in a pure shear geometry (Rivlin 1953). The stress-strain curves across five notched samples for each elastomer in FIG. 8C show that E1 and E2 have very different critical strains for crack propagation and T of E1 (113.0±9.7 J·m⁻²) is roughly an order of magnitude higher than Γ of E2 (11.5±3.7 J·m⁻²) (FIG. 8D). The impact of cyclobutane reactivity on toughness is almost the direct opposite of that observed in an end-linked system (FIG. 1A), where embedding similar mechanophores into the middle of chains leads to 8-fold lower tearing energy compared to the non-mechanophore control (FIG. 8E-8F) (Wang et al. J. Am. Chem. Soc. 143, 3714-3718 (2021)). Here, stitching polymer chains together through the much mechanically weaker reactant leads to a much tougher material.

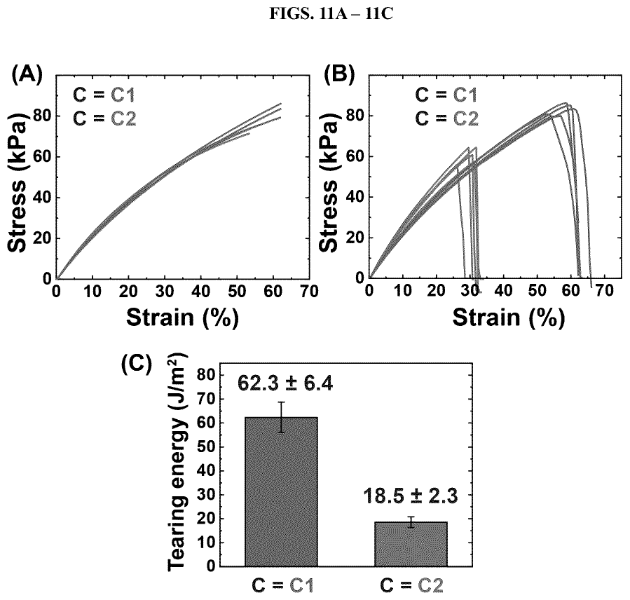

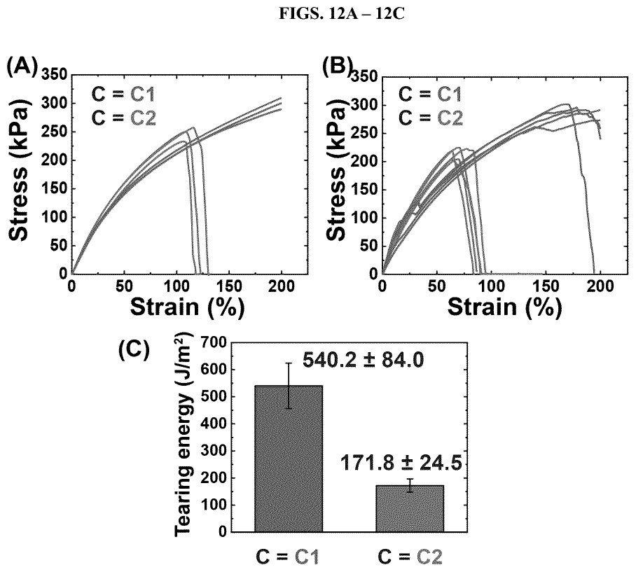

The enhanced toughness brought about by replacing C2 with C1 is not limited to networks made from a 2-methoxyethyl acrylate co-monomer, to the elastomer state, or to the specific characterization test—although the magnitude of the effect does change. For example, the same trend observed in tearing is also observed in fatigue testing, which minimizes contributions to the tearing resistance from the energy that is dissipated rather than stored elastically during network stretching (hysteresis) by slowing down the crack growth rate with cyclic loading (Lake et al. J. Appl. Polym. Sci. 9, 1233-1251 (1965); Tang et al. Extrem. Mech. Lett. 10, 24-31 (2017)). As shown in FIG. 8G-8H, both E1 and E2 have fatigue thresholds below their tearing energies (shaded areas), which is common for elastomers (Lake et al. Proc. R. Soc. London, Ser. A Math. Phys. Sci. 300, 108-119 (1967); Lake 1965). However, the fatigue threshold of E1 (52.0 J·m⁻²) is still much larger than that of E2 (8.0 J·m⁻²). Moreover, a similar toughening effect also exists when the 2-methoxyethyl acrylate networks are prepared as organogels (Γ=62.3±6.4 vs. 18.5±2.3 J·m⁻², FIGS. 11A-11C) and when elastomers are made from ethyl acrylate monomers (Γ=540±84 vs. 172±25 J·m⁻², FIGS. 12A-12C).

The results raise the important question of why the effect of introducing the same scission reaction in side-chain crosslinked networks is almost the exact opposite of what is observed in the end-linked networks of FIG. 1A. This toughening effect is not due to simple energy dissipation in the bulk, as both G′ and G″ are unchanged by the cross-linkers. It was hypothesized that the different response to the embedded reactivity originates from an important topological difference. In the end-linked networks of FIG. 1A (Wang et al. J. Am. Chem. Soc. 143, 3714-3718 (2021)), every load-bearing chain has a mechanophore, so increasing the reactivity of the mechanophore increases every scission probability to the same extent. Incorporating the mechanophore as the side-chain cross-linker, however, only weakens the cross-linker and not the primary chain.

### Example 5—Toughening Mechanism of Mechanochemically Weak Cross-Linkers

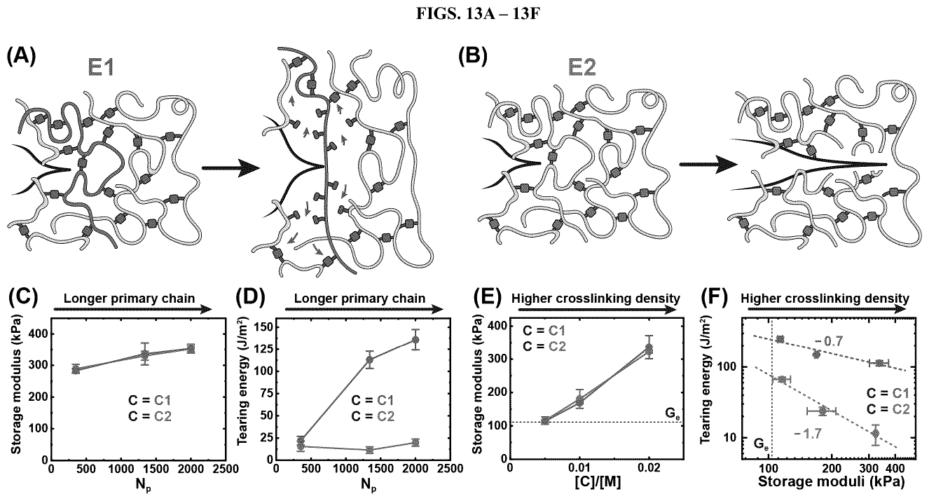

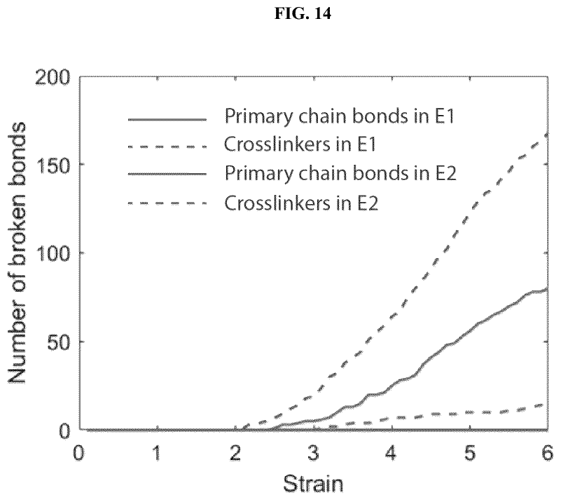

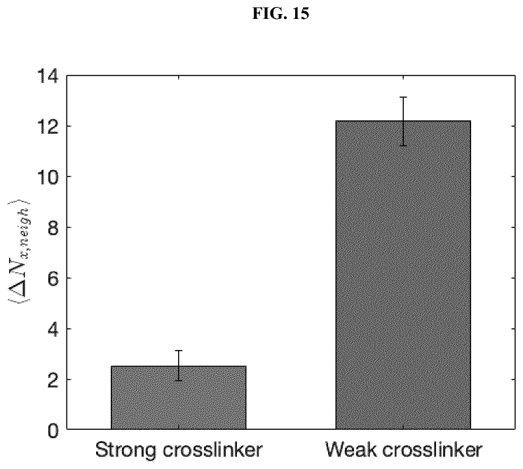

Toughening mechanism of E1. The consequences of the topology are proposed in FIG. 13A. referential scission through the cycloreversion of intra/intermolecular C1, rather than primary chain scission, lengthens rather than removes the bridging chain at the crack interface, which gives rise to the increase in tearing energy. There is no such preferential cross-linker reactivity in E2, because the cross-linkers are similarly as strong as the primary chain (FIG. 13B). A molecular dynamics simulation of network fracture under uniaxial stretching provides a consistent picture: (1) at high strain, mechanochemically weak cross-linkers in E1 break exclusively (up to a strain of 6) while leaving the primary chain bonds intact (FIG. 14) and (2) relevant elastically active strands between cross-linkers, on average, become much longer per bond scission event in E1 compared to that in E2 (FIG. 15).

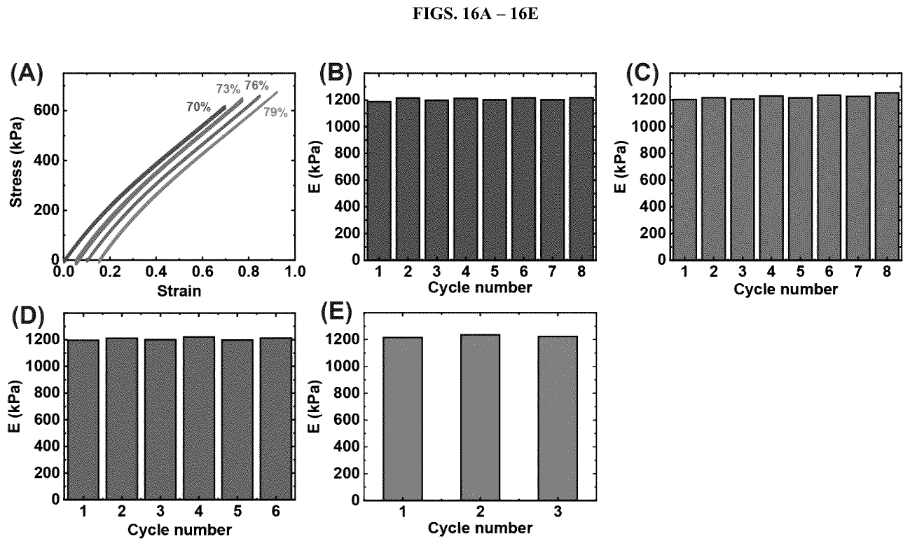

Random network cross-linking comprises a complex mixture of intramolecular and intermolecular junctions and loops (Wang et al. Chem. Sci. 10, 5332-5337 (2019)). The programmed cycloreversion of intramolecular C1 releases hidden length in a manner that is similar to noncovalent domain unfolding (Chung et al. Nat. Mater. 13, 1055-1062 (2014); Fang et al. Nat. Commun. 4, (2013)) and covalent reactive strand extension (RSE) (Wang et al. Science 374, 193-196 (2021)), while that of intermolecular C1 deviates the crack and increases pathway tortuosity. The effect of C1 is reminiscent of the toughening effects observed in most dynamic side-chain crosslinked network systems (Sun et al. Nature 489, 133-136 (2012); Neal et al. J. Am. Chem. Soc. 137, 4846-4850 (2015); Sakai et al. ACS Macro Lett. 9, 1108-1113 (2020); Du et al. Nat. Commun. 2022 131 13, 1-9 (2022)). The dynamic nature of the crosslinking makes networks formed by such “sacrificial bonds” prone to greater hysteresis and viscous energy dissipation during use. Although the magnitude of the overall toughening observed from the use of C1 is smaller than from the addition of weak, dynamic cross-linking (Sun 2012; Du 2022), it comes with negligible hysteresis (FIGS. 16A-16E) or contribution to loss modulus (FIG. 8A). It also provides a model system of fixed covalent structure that allows force-coupled crosslinker dissociation to be studied in the absence of other bond exchange processes.

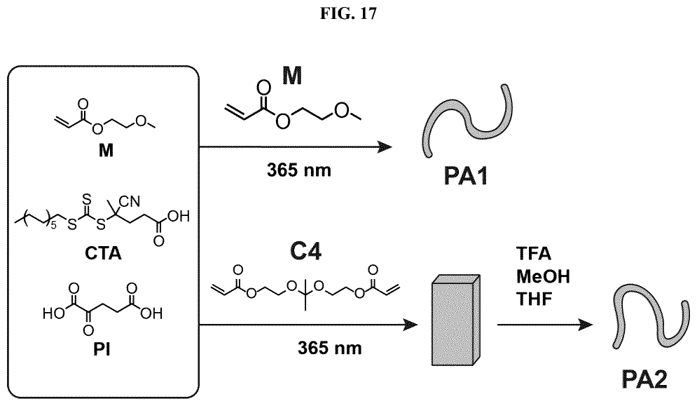

Based on the image in FIG. 13A, the contour length of the primary chain influences this toughening effect and so the synthetic control afforded by RAFT was used to vary the degree of polymerization of the primary chains (Nₚ) (Table 2) while keeping the extent of cross-linking constant. The Nₚ of different networks were verified experimentally by comparing primary chains synthesized without cross-linker to the primary chains obtained by degrading the networks that were prepared with chemically degradable cross-linker C4 under the same conditions (see FIG. 17 and detailed methods herein).

Elastomers of differing Nₚ were prepared with either C1 or C2 and the moduli and swelling were again indistinguishable between mechanophore and control networks for all Nₚ(FIG. 13C, FIGS. 4A-4D). The moduli increase modestly with Nₚ, as expected due to the decrease in elastically inactive dangling chain ends (Henkel et al. Macromol. Chem. Phys. 215, 182-189 (2014)). The tearing energies of E2 did not change much with Nₚ (FIG. 13D), as is typical of conventionally cross-linked elastomers, whose tearing energy is dominated by the length of the chain between crosslinks (Lake 1967), which is kept almost constant here. The tearing energies of E1, however, depend substantially on the contour length of the primary chain (FIG. 13D). When the primary chains are short (Nₚ≈350), elastomer E1 is only slightly tougher than E2, but as the primary chains lengthen, the tearing energies of E1 increase more than 4-fold for Nₚ≈2000. The unusual dependence of T on Nₚ in E1 is consistent with our model; the longer primary chains need a more tortuous path.

The reactivity-enabled toughening afforded by C1 provides a mechanism to mitigate an otherwise intrinsic tradeoff in polymer network optimization, namely the inverse correlation of modulus and toughness. At fixed Nₚ≈1400, increasing the cross-linker content ([C]:[M]=1:200, 1:100, 1:50) leads to high modulus materials. As expected, this increase in modulus is accompanied by a significant drop of T in the control elastomer E2, with a scaling exponent of −1.7 that is similar to that reported in other side-chain crosslinked systems (FIG. 13F) (Kim et al. Science 374, 212-216 (2021); Zheng et al. Extrem. Mech. Lett. 51, 101608 (2022)). The loss of toughness is attributed to the decreased chain length between cross-linkers (Lake 1967). By comparison, the reactivity engineered into E1 enables the elastomer to acquire higher stiffness without sacrificing the same extent of tear resistance (scaling exponent of −0.7), because the loss in toughness due to higher cross-link density is offset by the enhanced toughness afforded by additional mechanophores.

The decrease of T for E1 with higher cross-linking (FIG. 13F) suggests that the primary chains cannot be completely pulled out before they break, because if tearing energy is dominated by complete chain pull-out (breaking all the C1 at least along one direction), then the tearing energy would increase while the modulus increases, as there are more cross-linkers to break to dissipate energy. This incomplete primary chain pull-out might originate from entanglement lockup under higher tension (Zheng et al. Macromol. Rapid Commun. 2200159 (2022)). The locked entanglements act like strong cross-linker C2, which force the primary chain to break instead of being pulled out. The length scale between two locked entanglements seems to be crosslinking density dependent. This hypothesis of incomplete chain pull-out suggests that the tearing energy of E1 would eventually saturate if Nₚ keeps increasing while keeping crosslinking density constant because the effective contour length between locked entanglements would limit the toughening effect (id.). The leveling off of tearing energy for E1 observed in FIG. 13D also supports this hypothesis.

### Example 6—Effect of Cross-Linker Mechanochemical Reactivities on Γ

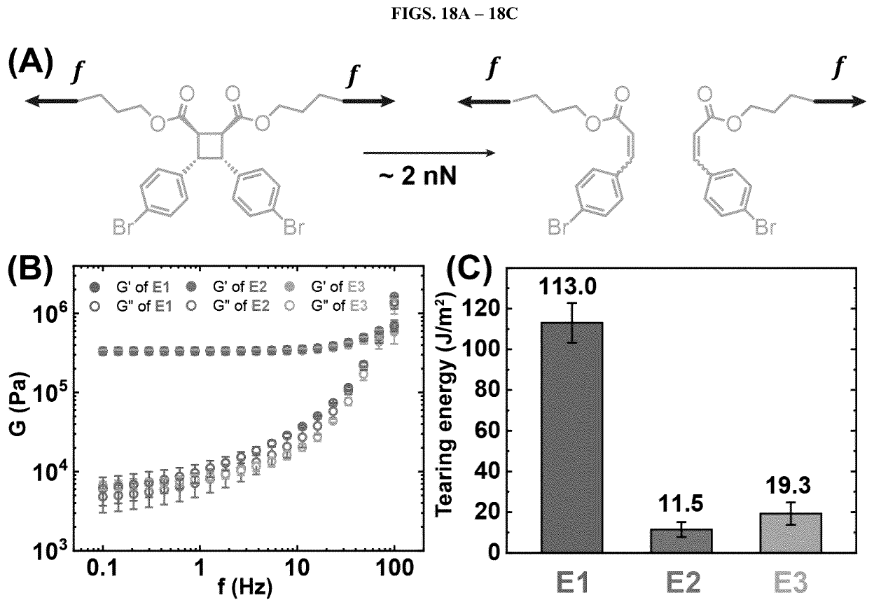

How the reactivity of the cross-linker, relative to the primary chain, influences the effect of cross-linker mechanochemical reactivities on I′ was postulated. Another mechanophore cross-linker C3 of intermediate strength (FIGS. 18A-18C) was synthesized; relevant force at break is roughly half that of conventional polymer components and just over twice that of C1). E3 results in only a very modest toughening effect (factor of <2 vs. >9 for otherwise identical networks; (see FIGS. 18A-18C), which is likely because the larger mechanochemical strength of C3 compared to C1 suppresses the lengthening efficiency of bridging chains before primary chains break. This result suggests that further decrease in the mechanochemical strength of the cross-linker while other components remain unchanged might potentially lead to tougher networks, yet such a cross-linker could suffer from poor thermal stability, which risks the physical integrity of the networks. The extreme case is the cross-linker has no strength (i.e., no cross-linker) or very low cross-linking density. Although, depending on the loading rate, such highly entangled systems could achieve high tearing energy through non-specific near-crack dissipation, their stiffness is no longer tunable with cross-linkers (Kim 2021; Zheng et al. Extrem. Mech. Lett. 51, 101608 (2022)).

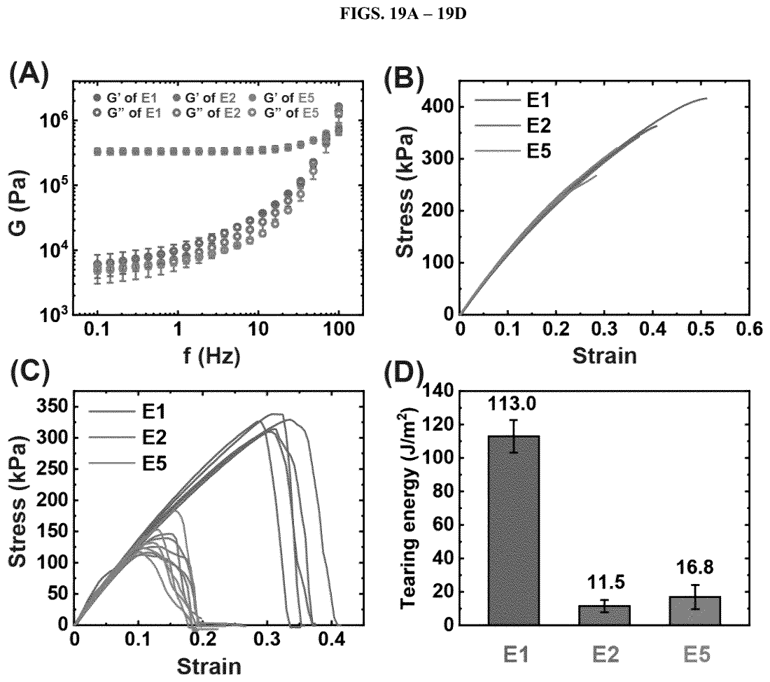

Finally, a cross-linker that reproduces the mechanochemical reactivity of C1, but which leads to molecular extension rather than scission, was tested. A cross-linker C5 (synthetic details provided in the supplementary materials), in which initial cyclobutane scission analogous to that found in C1 leads to the release of local stored length, but complete scission of the cross-linker needs subsequent homolytic bond scission in a manner analogous to C2 was synthesized. The new cross-linker C5 was incorporated into elastomer E5 following the same procedure as employed to make E1 and E2. Consistent with the models proposed above, E5 displays at best a very modest toughening effect compared to E2 (FIGS. 19A-19D), the difference in tearing energies is within the uncertainty of the measurement error). This result further supports the physical picture proposed in FIG. 13A, because C5 is mechanochemically strong after cycloreversion, which acts effectively as a strong crosslinker that is reminiscent of C2.

### Example 7—Elastomers Made from “Intermediate” Crosslinker C3

Results presented above suggest that the crosslinker needs to be weaker than the primary chain to achieve this toughening effect, but it is not known what relative strength between the crosslinker and the primary chain could give maximum effect. An “intermediate” crosslinker C3 (synthesis of C3 is shown in the section chemical synthesis) was synthesized based on a precursor of C1. Albeit no direct SMFS characterization on this specific cyclobutane, similar cis-diester substituted cyclobutanes characterized by SMFS show activation forces of ˜2 nN on the timescales of milliseconds (Wang et al. Nat. Commun. 7, 13433 (2016)), making C3 still a preferential site for breaking in networks compared to the primary chains. Elastomer E3 was made from C3 in the same way as E1 and E2, and they have similar shear moduli and stress-strain curves (FIG. 18B). However, the tearing energy of E3 (19.3±5.5 J·m⁻²) is only slightly larger than that of E2 and much smaller than that of E1. Therefore, to achieve a significant enhancement in tearing energy, the crosslinker is not just mechanochemically weak, but it has to be at least two times weaker than the primary chain. Another extreme would be the case when the crosslinkers need zero force to break (no crosslinker), which clearly cannot toughen the network as the polymer melt synthesized at the same condition flows instead of fractures. With the same primary chain length and crosslinking density, an optimal activation force is expected for the crosslinkers that can provide the most significant toughening effect, but it needs a more sophisticated design of the crosslinker to achieve.

### Example 8—Degree of Polymerization of Primary Chains Nₚ in Elastomers

To show that the chain transfer agent has good control over the molecular weight of primary chains in the networks, an experiment shown in FIG. 17 was performed. The same pre-gel solution was separately polymerized with an acid-degradable crosslinker C4 and the same amount of additional monomer, such that the concentrations of acrylate groups are identical in these two mixtures. Polymerizations were performed as described herein. Polymer PA1 was polymerized without crosslinker, polymer PA2 was obtained by first forming elastomer E4 and then degrading the crosslinkers. After polymerization, 200 mg PA1 and 200 mg E4 were separately submerged and stirred in a solution containing trifluoroacetic acid (TFA) (100 μL) and methanol (200 μL) in 10 mL THF. Elastomer E4 first swelled to equilibrium and degraded within 5 hours. After 24 hours, PA1 and PA2 were obtained by extracting with EA and washing with water.

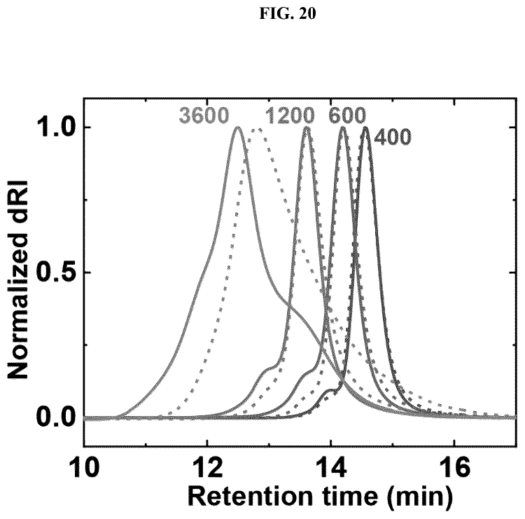

GPC traces of PA1 and PA2 prepared with 4 different stoichiometries are shown in FIG. 20. When [M]/[CTA]≤1200, CTA has good control over the molecular weights of primary chains (solid and dashed lines are almost overlapped for red, blue, and black). When [M]/[CTA]=3600, the PA2 synthesized from degrading the network has a retention time longer than PA1 synthesized without crosslinkers (green).

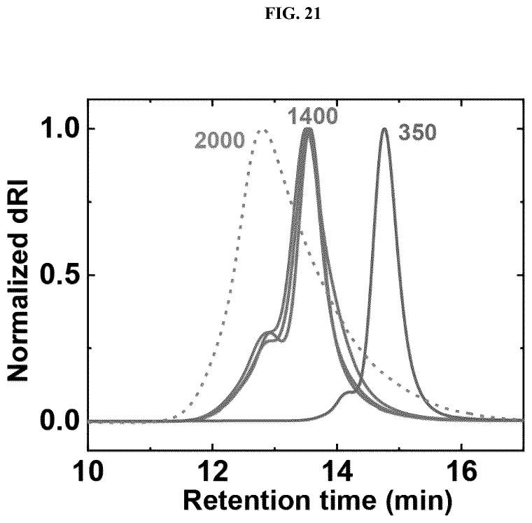

Elastomers reported in FIGS. 13C-13F and FIG. 6 and Table 1, were prepared at three different [M]: [CTA] ratios, which are 300:1, 1200:1, and 3600:1. The same pre-gel solution was used and prepared PA1 and PA2 in parallel with E1 and E2. Based on FIG. 20, the molecular weight of PA1 was used as an estimate for Nₚ when [M]/[CTA]≤1200, and the molecular weight of PA2 was used to estimate Nₚ when [M]/[CTA]=3600. In the end, three different [M]: [CTA] ratios resulted in Nₚ˜ 350, 1400, and 2000, respectively. GPC traces for the primary chains that were used for Nₚ estimation are shown in FIG. 21. A summary of molecular weights and estimated Nₚ are shown in Table 2.

TABLE 2
Summary of Nₚ estimation for elastomers in FIG. 13 and FIG. 6.
Data in the first, second, and third rows correspond to the blue,
red, and dashed green traces shown in FIG. 20, respectively.

| | [M]:[C] | | |
|---|---|---|---|
| [M]:[CTA] | 200:1 | 100:1 | 50:1 |
| 300 | — | — | Mₙ = 45.0 ± 0.9 kDa |
| | | | Nₚ ≈ 350 |
| 1200 | Mₙ = 207.9 ± 8.3 kDa | Mₙ = 163.2 ± 3.2 kDa | Mₙ = 174.1 ± 3.5 kDa |
| | Nₚ ≈ 1590 | Nₚ ≈ 1260 | Nₚ ≈ 1340 |
| 3600 | — | — | Mₙ = 260 ± 14 kDa |
| | | | Nₚ ≈ 2000 |

### Example 9—Cyclic Loading of E1

To investigate whether the C1 can be activated by cyclic tensile loading close to the strain-at-break, cyclic loading tests were performed to maximum strains of 0.7, 0.73, 0.76, and 0.79 for E1 prepared with a stoichiometry of [M]: [C]: [CTA]: [PI]=1:1/50:1/1200:1/2000. The results are shown in FIGS. 16A-16E.

### Example 10—Molecular Dynamic Simulation of Tensile Fracture

General simulation details. The Large-scale Atomic/Molecular Massively Parallel Simulator (LAMMPS) package (Plimpton et al. J. Comput. Phys. 117, 1-19 (1995)) was used with the coarse-grained bead-spring model (Grest et al. Phys. Rev. A 33, 3628 (1986); Kremer et al. J. Chem. Phys. 92, 5057 (1990)) of polymer chains. Monomers (beads) are interacting via the Lennard-Jones (LJ) potential (Johnson et al. Mol. Phys. 78, 591-618 (1993); Frenkel, D. & Smit, B. Understanding Molecular Simulation: Monte Carlo Simulations in Various Ensembles. (Academic Press, 2002)).

$${U}_{LJ} ( r ; {r}_{c} ) = \{ \begin{matrix}4 ε [ {( \frac{σ}{r} )}^{12} - {( \frac{σ}{r} )}^{6} - {( \frac{σ}{{r}_{c}} )}^{12} + {( \frac{σ}{{r}_{c}} )}^{6} ] & r \leq {r}_{c} \\ 0 & r > {r}_{c}\end{matrix} \qquad ( S1 )$$

where the LJ interaction is truncated at r_{c}=2.5σ. We set ε=1 kT, which corresponds to non-solvent condition of implicit solvent. Bonded interactions are modeled by the Finite Extensible Nonlinear Elastic (FENE) potential (Kremer 1990)

$${U}_{FENE} ( r ) = \{ \begin{matrix}- \frac{1}{2} {KR}_{0}^{2} ln ⌊ 1 - {( \frac{r}{{R}_{0}} )}^{2} ⌋ & r \leq {R}_{0} \\ ∞ & r > {R}_{0}\end{matrix} \qquad ( S2 )$$

with a spring constant K=30 kTσ⁻², and a cutoff distance R₀=1.5σ. The total bond interaction potential also includes a purely repulsive LJ term,

U_{b}(r)=U_{FENE}(r)+U_{LJ}(r;r_{c}) (S3)
where the LJ cutoff is r_{c}=2^{1/6}σ.

In all simulations, the pressure was held constant (at P=0) using the Nose-Hoover barostat with a damping parameter of 100 τ_{LJ}. The temperature was held constant (kT=1 in Lammps units) using the Nose-Hoover thermostat with a damping parameter of 10 τ_{LJ}. All molecular dynamics simulations were performed with an integration time-step of 0.01 τ_{LJ}.

Network Formation. A precursor melt of M=400 chains with n=64 beads each was equilibrated in the NPT ensemble with P=0 by simulating a long trajectory, 10⁶τ_{LJ}, which corresponds to ˜10²τ_{R}, where τ_{R}˜10⁴τ_{LJ} is the relaxation time of a chain (Halverson, J. D., Lee, W. B., Grest, G. S., Grosberg, A. Y. & Kremer, K. Molecular dynamics simulation study of nonconcatenated ring polymers in a melt. I. Statics. J. Chem. Phys. 134, 204904 (2011)). The equilibrated melt simulations were instantaneously randomly crosslinked emulating gamma irradiation crosslinking (Leibler et al. Phys. Rev. Lett. 55, 1110 (1985); Grest et al. Macromolecules 23, 4994-5000 (1990)), A snapshot of the equilibrated melt was vulcanized by randomly selecting and bonding monomer pairs at a distance below 1.3σ, which corresponds to the first peak in the melt radial distribution function. The extent of reaction of this vulcanization process was p=0.1. The only restriction imposed on the selection of pairs of monomers for bonding was to exclude any monomers at a curvilinear distance of s=1,2 bonds away from an existing crosslinked monomer, thereby setting the minimal strand length to 2 and the minimum loop length to 4. The networks were equilibrated for 10⁵τ_{LJ} at the NPT ensemble at P=0, followed by a production run of 9·10⁵τ_{LJ}. The average size of the simulation box was determined from the production run and used for the ensuing pulling simulations.

Network topology. The analysis of the vulcanized network topology was carried out using our in-house code that is based on the analogy between elasticity and electrical resistivity (Gennes et al. J. Phys. Lettres 37, 1-2 (1976)). The network is mapped onto a network of nodes (formed by two monomers bonded by a crosslink) and edges (all strands connected to these nodes). The NetworkX python package (Hagberg et al. in Proceedings of the 7th Python in Science Conference (SciPy 2008) (2008)) is used for identifying the basis set of loops describing the network (Paton, ACM 12, 514-518 (1969)). In the following step, the network is modeled as a collection of resistors (resistivity of strands is proportional to Nₓ⁻¹) connected at nodes (crosslinks). The voltage of one strand in the largest loop is then set to some finite values and the Kirchhoff circuit laws are then applied by requiring 1) the sum of all currents passing through a node to be zero and 2) the voltage difference along any closed loop is zero. The numerical solution of this set of equations yields the current in each strand in the network, whereby those strands that carry current as elastically active strands are identified.

The topology analysis was applied to the network formed by random crosslinking as described above. The gel fraction of this network is over 99%, with 66% of the mass being elastically active. The average length (i.e., number of bonds) of the elastically active strands is [symbol] Nₓ [symbol] =11.7.

Calibrating breakable bonds. In the bond scission simulations, the unbreakable FENE bond potentials were replaced by the Morse potential (Morse, Phys. Rev. 34, 57 (1929)) for the breakable bonds,

$${U}_{morse} ( r ) = {{D}_{e} ( 1 - {e}^{- a ( r - {r}_{e} )} )}^{2} \qquad ( S4 )$$

where the parameters Dₑ determines the energy of dissociation, rₑ is the equilibrium bond length and a controls the width of the potential. For the strong bonds, Dₑ=120 kT was chosen and, for the weak bonds, Dₑ=30 kT was chosen following the experimental system where the strength of the strong and weak bonds differs by a factor of ˜4. To keep the equilibrium properties of the bonds the same upon switching from unbreakable to breakable bonds, the parameters of the Morse potential were calibrated to match the FENE potential used in the preparation of the networks, Eq. S3. The parameters

$${r}_{e} = 0.96 σ , a = \sqrt{\frac{31.52}{2 {D}_{e}}} \qquad ( S5 )$$

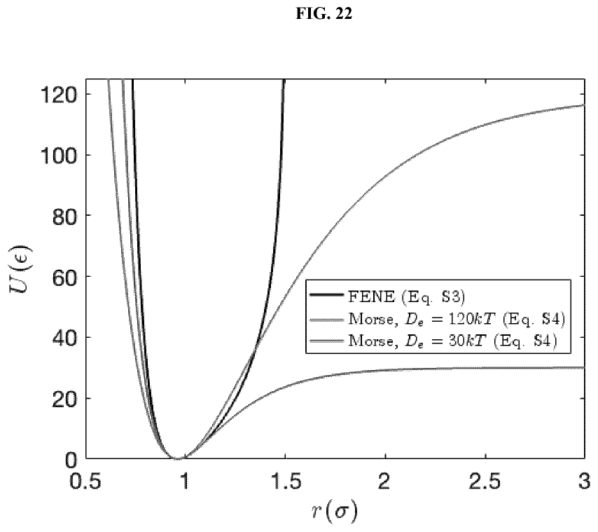

were chosen to match the equilibrium length and the local stiffness around the equilibrium of the FENE potential (second derivative of bond potential at equilibrium length U″(rₑ)). The bonded potentials used are shown in FIG. 22, comparing the FENE and Morse potentials.

Two networks were simulated with these two bond potentials: (i) A strong crosslinker network with both primary chain bonds and crosslinker Dₑ=120 kT; (ii) A weak crosslinker network with primary chain bonds having Dₑ=120 kT and crosslinker Dₑ=30 kT.

Simulations of bonds scission upon uniaxial extension. The equilibrated networks are uniaxially stretched by changing the z dimension of the simulation box, L_{z}, from its unperturbed average size, L_{z,0}, while the other two dimensions are relaxed at zero pressure by the Nose-Hover barostat. This mimics the strain-control uniaxial stretching experiments. The network is stretched at a constant strain rate of 10⁻⁵τ⁻¹ up to the strain (L_{z}−L_{z,0})/L_{z,0}=6. As shown in FIG. 14, the bond scission in weak crosslinker network (blue) almost exclusively occurs at crosslinkers (blue dashed line), while the primary chain bonds are mostly intact (blue solid line). This effect is not observed in the strong crosslinker network (red), in which the primary chain bonds (red solid line) break much more than the crosslinker bonds (red dashed line).

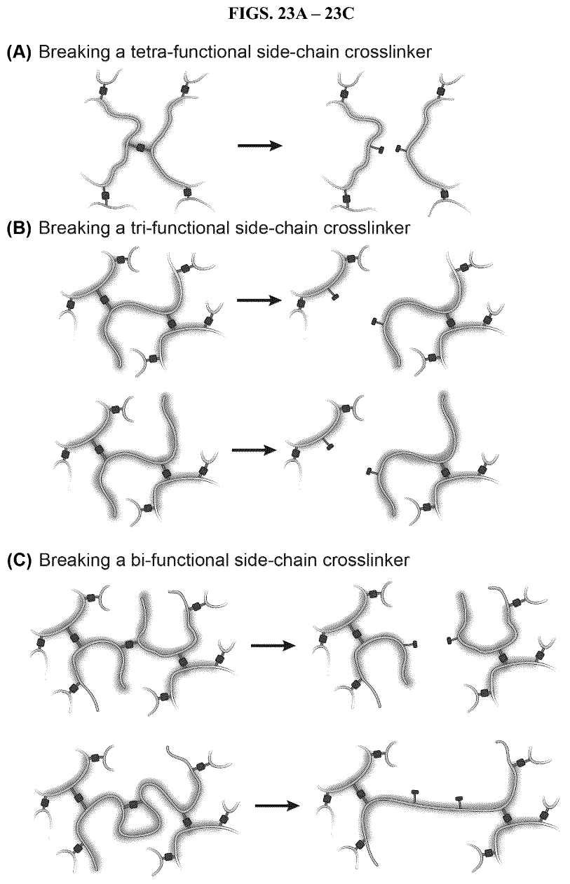

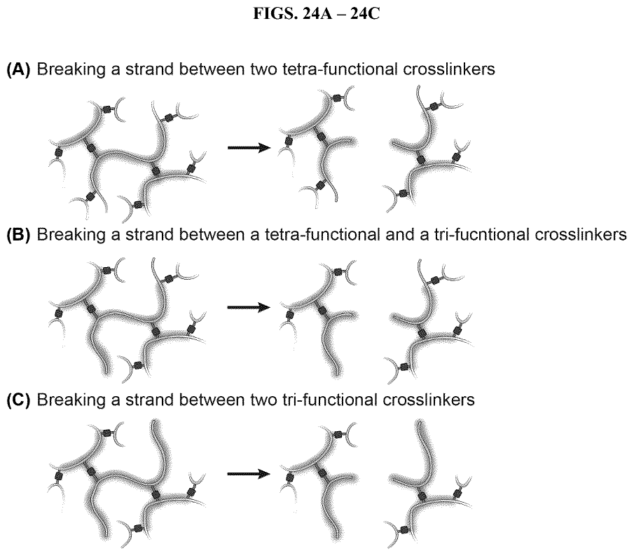

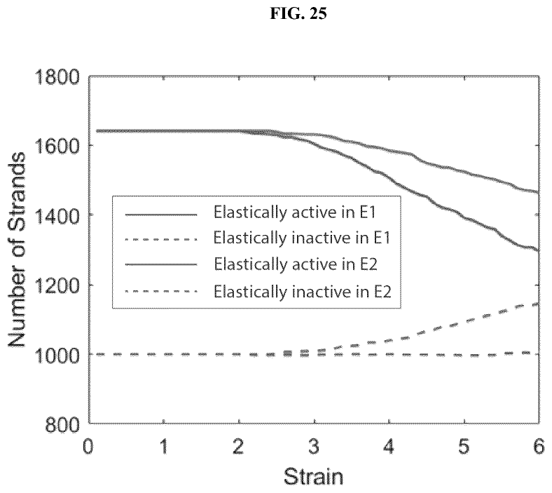

Effect of bond breaking. The effect of breaking a bond on the change of average strand lengths [symbol] ΔN_{x,neigh} [symbol] that are affected by the bond rupture event (neighboring strands of a broken bond) is shown. If the broken bond is a crosslinker, the neighboring strands are defined as the four strands it is connected to. If the broken bond is a primary chain bond, the neighboring strands are defined as the strand it is located on and all other strands directly connected to this strand. The definition of neighboring strands associated with a broken bond is shown in FIGS. 23A-23C (crosslinkers) and FIGS. 24A-24C (primary chain bonds) for different scenarios. As shown in FIG. 25, in a weak crosslinker network where the broken bonds are all crosslinkers, the number of elastically inactive strands does not change, while the elastically active strands are combined into longer strands, and their number decreases. In contrast to this result, for the strong crosslinker network where the broken bonds are dominated by the primary chains, the overall effect is an increase in the number of inactive strands and a decrease in the number of active strands as elastically active strands are broken into elastically inactive strands.

The distinct effect of breaking either primary chain bonds or crosslinkers and the dominance of these two types of bonds breakage (primary chain bonds or crosslinkers, FIG. 22) in the two types of networks leads to different modifications on the network topology after bond breaking. FIG. 15 shows the change in average neighboring strand length [symbol] ΔN_{x,neigh} [symbol] due to a broken bond. For the weak crosslinker network, the change of neighboring strand length is about the average strand length of the network (Nₓ), while the change in the strong crosslinker network is only ˜ [symbol] Nₓ [symbol] /5. These effects will be magnified for the bridging strand near the crack tip where multiple crosslinkers on its primary chain could break and its length grows by [symbol] Nₓ [symbol] for every broken crosslinker. According to the Lakes-Thomas theory, the fracture energy is proportional to the length of these bridging strands. Therefore, the fracture energy of the weak crosslinker network will be increased by breaking crosslinkers in comparison to the strong crosslinker network where the breaking bridging strands are relatively shorter ˜ [symbol] Nₓ

[symbol]

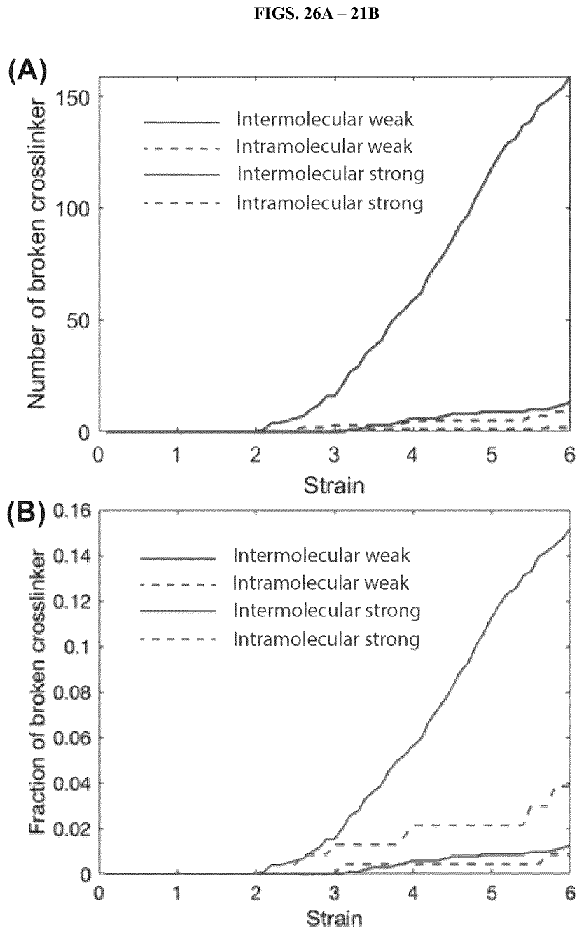

Inter- vs. intra-molecular crosslinkers. In the simulated network, the number of intermolecular crosslinkers is 4.5 times higher than the intramolecular ones. As shown in FIGS. 26A-26B, selective scission of intermolecular crosslinkers was found during deformation in the network made from weak crosslinkers. The fraction of broken intermolecular weak crosslinkers is much higher in comparison with intramolecular weak crosslinkers during scission, indicating a much higher tendency towards breaking intermolecular weak crosslinkers. It is acknowledged that the specific composition of our networks, with “4.5 times more intermolecular than intramolecular crosslinkers,” is unique to our simulation. Therefore, the tendency to break intermolecular weak crosslinkers may vary with changes in network composition. For the composition used in our simulation, such selective scission of intermolecular crosslinker in networks made from strong crosslinkers was not observed.

### Discussion

When considered in combination with quantitative relationships between mechanophore activity and material properties (Wang et al. J. Am. Chem. Soc. 143, 3714-3718 (2021); Wang et al. Science 374, 193-196 (2021)), the work disclosed herein accentuates how the position (along the polymer chain vs. at the cross-links), type (scissile vs. non-scissile), and mechanochemical reactivity of mechanophores work in combination to dictate the mechanical properties of the networks in which they are incorporated. When mechanophores are incorporated along every stress-bearing polymer strand within a network, there is a direct correlation between mechanochemical reactivity and network toughness—the easier it is to break the mechanophore, the easier it is to tear the network (Wang et al. J. Am. Chem. Soc. 143, 3714-3718 (2021)). Replacing strands of scissile mechanophores with analogous strands of non-scissile mechanophores, however, leads to a toughening effect (Wang et al. Science 374, 193-196 (2021)), because the latter release “hidden length” rather than breaking, and the enhanced properties at the single strand level are translated to the macroscopic materials. When mechanophores are incorporated as otherwise conventional side-chain cross-linkers, however, the responses are opposite. Mechanochemically more reactive (weaker) mechanophores lead to tougher rather than more brittle networks (FIG. 8D), and scissile mechanophores outperform their non-scissile analogues (FIGS. 19A-19D), because the former can share the load along the primary chains through cycloreversion, while the latter cannot as they are as strong as the primary chain after activation.

From these observations, the macroscopic properties of polymer networks can be directly correlated with molecular reactivity and mechanisms. Fracture in cross-linked networks is productively viewed as a sort of “molecular composite,” and that picture has implications for the use of “weak” bonds to toughen covalent polymer networks. Typically, toughening a network through weak cross-linkers involves the use of dynamic interactions, such as ionic bonding (Sun 2012), hydrogen bonding (Neal 2015), the reversible formation of stable radicals (Sakai 2020; Sakai 2020; Watabe et al. Macromolecules 55, 5795-5802 (2022)), or dynamic covalent bonding (Du 2022). Without introducing non-dynamic covalent cross-linkers, all of these systems would eventually lead to a fluid network structure and added, potentially undesirable viscous dissipation on longer timescales or at higher temperatures. The capacity to recombine and/or exchange during or following the scission of dynamic bonds also complicates their toughening mechanism even in hybrid networks that use partial covalent cross-linking to provide long-term shape persistence (Sun 2012; Miquelard-Garnier et al. Polymer 50, 481-490 (2009); Long et al. Macromolecules 47, 7243-7250 (2014)). No such scrambling or reformation is accessible in E1 because C1 decouples its labile mechanochemical reactivity from thermal reversibility. These results demonstrate that preferential bond scission at the propagating crack front alone is sufficient to provide a substantive toughening effect. It is noted that pathway tortuosity at the macroscale, for example in composites, is an established toughening mechanism (Ritchie et al. Int. J. Fract. 100, 55-83 (1999)), and the results observed here indicate that molecular analogues of that behavior likely contribute to the more complex toughening mechanisms at play in reversible networks. For both those dynamic networks and the static covalent networks demonstrated here, the primary chain length effect offers a clear design principle for optimizing reactivity-enabled toughening without losing stiffness.

Toughening effects can also be obtained through other mechanisms, such as dynamic bonding and entanglements. An advantage of toughening through the programmed reactivity of covalent cross-linkers is that the properties of the mechanophore network are indistinguishable from those of the conventional network, with the exception of the preferential scission behavior that occurs only when and where necessary to inhibit material tearing and fatigue; even the primary chain effect has a very modest impact on low-strain mechanical properties relative to the impact on toughness. The utility of C1 shows that these gains can be realized with mechanophores of good thermal stability (Zhang 2017) (FIGS. 9A-9E).

1. An acrylate polymer comprising a moiety of formula (I) or a moiety of formula (II): 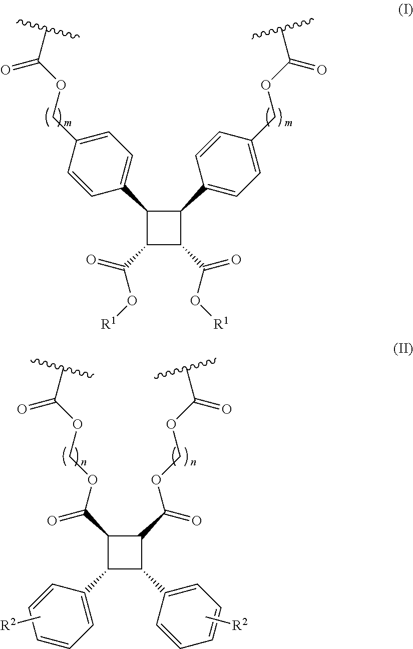
wherein:
R¹ is selected from C₁-C₁₂ alkyl and hydrogen;
R² is selected from hydrogen, halo, C₁-C₆ alkyl, C₁-C₆ haloalkyl, cyano, and nitro;
m is 1, 2, 3, 4, 5, or 6; and
n is 1, 2, 3, 4, 5, or 6.

2. The acrylate polymer of claim 1, comprising a moiety of formula (I).

3. The acrylate polymer of claim 2, wherein R¹ is methyl.

4. The acrylate polymer of claim 2, wherein m is 4.

5. The acrylate polymer of claim 2, wherein the moiety of formula (I) is:

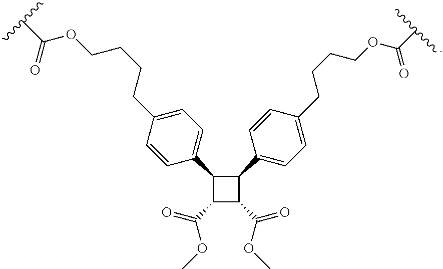 [SMILES: *C(=O)OCCCCc1ccc([C@@H]2[C@@H](C(=O)OC)[C@@H](C(=O)OC)[C@@H]2c2ccc(CCCCOC(*)=O)cc2)cc1]

6. The acrylate polymer of claim 1, comprising a moiety of formula (II).

7. The acrylate polymer of claim 6, wherein R² is bromo.

8. The acrylate polymer of claim 6, wherein n is 4.

9. The acrylate polymer of claim 6, wherein the moiety of formula (II) is:

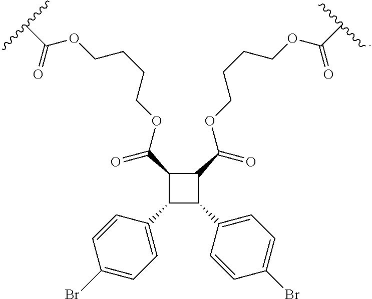

10. The acrylate polymer of claim 1, comprising at least one (meth)acrylate monomer selected from an alkyl (meth)acrylate, a hydroxyalkyl (meth)acrylate, an alkoxyalkyl (meth)acrylate, a cycloalkyl (meth)acrylate, and an aromatic (meth)acrylate.

11. The acrylate polymer of claim 10, comprising an alkyl (meth)acrylate monomer selected from methyl (meth)acrylate, ethyl (meth)acrylate, n-propyl (meth)acrylate, n-butyl (meth)acrylate, iso-butyl (meth)acrylate, tert-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, and stearyl (meth)acrylate.

12. The acrylate polymer of claim 10, comprising a hydroxyalkyl (meth)acrylate monomer selected from hydroxymethyl (meth)acrylate, 2-hydroxyethyl (meth)acrylate, 3-hydroxypropyl (meth)acrylate, and 4-hydroxybutyl (meth)acrylate.

13. The acrylate polymer of claim 10, comprising an alkoxyalkyl (meth)acrylate selected from 2-methoxyethyl (meth)acrylate, 2-ethoxyethyl (meth)acrylate, 1-methyl-2-methoxyethyl (meth)acrylate, ethylene glycol methyl ether (meth)acrylate, diethylene glycol methyl ether (meth)acrylate, and triethylene glycol methyl ether (meth)acrylate.

14. The acrylate polymer of claim 10, comprising a cycloalkyl (meth)acrylate selected from cyclopentyl (meth)acrylate, cyclohexyl (meth)acrylate, 2-cyclohexylethyl (meth)acrylate, 3-cyclohexylpropyl (meth)acrylate, 2-norbornyl (meth)acrylate, and isobornyl (meth)acrylate.

15. The acrylate polymer of claim 1, comprising at least two different (meth)acrylate monomers.

16. A compound of formula (III) or formula (IV): 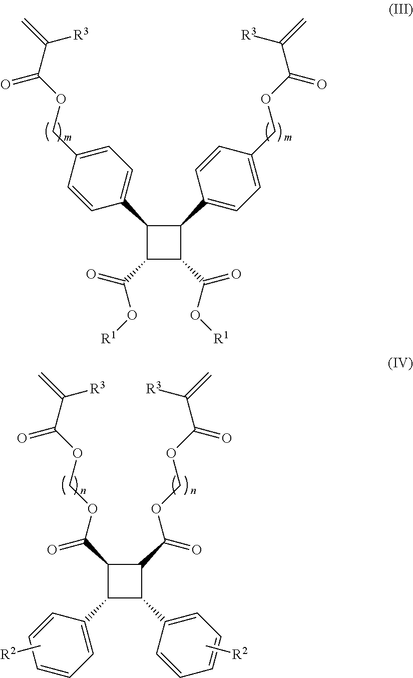
wherein:
R¹ is selected from C₁-C₁₂ alkyl and hydrogen;
R² is selected from hydrogen, halo, C₁-C₆ alkyl, C₁-C₆ haloalkyl, cyano, and nitro;
R³ is selected from hydrogen and methyl;
m is 1, 2, 3, 4, 5, or 6; and
n is 1, 2, 3, 4, 5, or 6.

17.-26. (canceled)

27. An acrylate polymer prepared by:
(a) controlled radical polymerization of a first mixture comprising a (meth)acrylate monomer, to form a pre-gel mixture comprising acrylate polymer chains; and
(b) crosslinking the acrylate polymer chains in the pre-gel mixture with the compound of claim 16.

28. The acrylate polymer of claim 27, wherein the controlled radical polymerization is reversible addition-fragmentation chain transfer (RAFT).

29.-31. (canceled)

32. A method of toughening an acrylate-based polymeric material, comprising:
incorporating a moiety of formula (I) or formula (II) into the acrylate-based polymeric material: 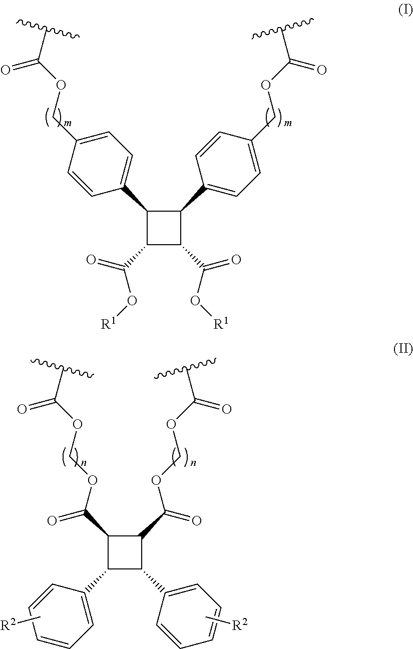
wherein:
R¹ is selected from C₁-C₁₂ alkyl and hydrogen;
R² is selected from hydrogen, halo, C₁-C₆ alkyl, C₁-C₆ haloalkyl, cyano, and nitro;
m is 1, 2, 3, 4, 5, or 6; and
n is 1, 2, 3, 4, 5, or 6.

33. The method of claim 32, wherein the incorporating step comprises:
(a) polymerizing a (meth)acrylate monomer to form a pre-gel mixture;
(b) adding a compound of formula (III) or formula (IV) to the pre-gel mixture to form a second mixture: 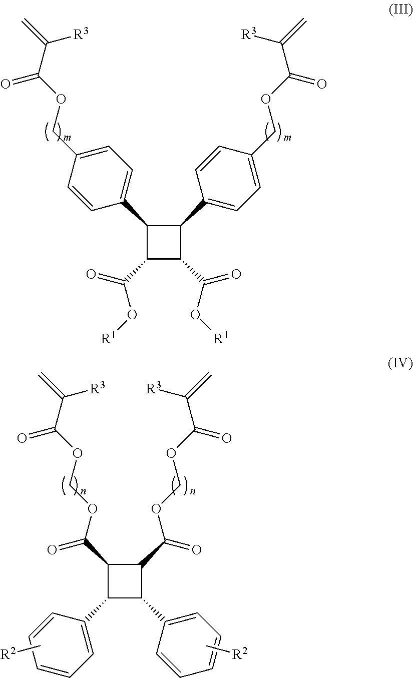
wherein:
R¹ is selected from C₁-C₁₂ alkyl and hydrogen;
R² is selected from hydrogen, halo, C₁-C₆ alkyl, C₁-C₆ haloalkyl, cyano, and nitro;
R³ is selected from hydrogen and methyl;
m is 1, 2, 3, 4, 5, or 6; and
n is 1, 2, 3, 4, 5, or 6; and
(c) exposing the second mixture to light to incorporate the moiety of formula (I) or formula (II) into the acrylate-based polymeric material and form the toughened acrylate-based polymeric material.

34.-36. (canceled)
